(19) United States
(12) Patent Application Publication
(10) Pub. No.: US 2012/0020960 A1
(43) Pub. Date: Jan. 26, 2012

(54) Thymic Stromal Lymphopoietin (TSLP) and OX40 Ligand in Cancer

(75) Inventors: Anna Karolina Palucka, Dallas, TX (US); Jacques F. Banchereau, Dallas, TX (US)

(73) Assignee: Baylor Research Institute, Dallas, TX (US)

(21) Appl. No.: 13/189,244

(22) Filed: Jul. 22, 2011

Related U.S. Application Data
(60) Provisional application No. 61/367,743, filed on Jul. 26, 2010.

(51) Int. Cl. A61K 39/395; A61P 35/00; A61P 29/00; A61K 38/16; C07K 16/24

(52) U.S. Cl. 424/133.1; 424/158.1; 424/178.1; 514/7.6; 424/145.1; 530/389.2; 530/391.1; 530/387.3

(57) ABSTRACT

Compositions and methods for an immunotherapeutic approach for human breast cancer is provided herein. Any antagonist of thymic stromal lymphopoietin (TSLP) and/or OX40L to inhibit tumor development and IL-13 secretion by blocking the upregulation of OX40L by DCs exposed to breast cancer, thereby blocking their capacity to generate inflammatory IL-13⁺TNFα⁺IL-10^{neg}CD4⁺ T cells (Th2 cells). Thus, TSLP, and/or down-stream pathways, represent novel potential therapeutic targets against human breast cancer.

### CROSS-REFERENCE TO RELATED APPLICATIONS

This patent application is a non-provisional application of U.S. Provisional Patent Application No. 61/367,743 filed on Jul. 26, 2010 and entitled “Thymic Stromal Lymphopoietin (TSLP) and OX40 Ligand in Cancer,” which is hereby incorporated by reference in its entirety.

### STATEMENT OF FEDERALLY FUNDED RESEARCH

This invention was made with U.S. Government support under Contract No. AI057234 awarded by the NIH. The government has certain rights in this invention.

### TECHNICAL FIELD OF THE INVENTION

The present invention relates in general to the field of cancer treatment, and more particularly, to treating cancers of epithelial origin by inhibiting or neutralizing thymic stromal lymphopoietin (TSLP) and/or OX40L to inhibit tumor development and IL-13 secretion.

### REFERENCE TO A SEQUENCE LISTING

None.

### BACKGROUND OF THE INVENTION

Without limiting the scope of the invention, its background is described in connection with the role of thymic stromal lymphopoietin (TSLP) in disease diagnosis and treatment.

U.S. Pat. No. 7,709,217, issued to Lyman et al. is directed to modified human thymic stromal lymphopoietin, including, modified, furin resistant human TSLP polypeptides and polynucleotides encoding the modified human TSLP polypeptides. Pharmaceutical compositions, B and T cell activation agents, assays and methods of use are also described.

U.S. Patent Application Publication No. 2007/0237787 (Leonard et al. 2007) discloses methods for specifically inducing proliferation of CD4+ T cells. The methods are of use in treating immunodeficiencies, such as an immunodeficiency produced by infection with an immunodeficiency virus, such as infection with a human immunodeficiency virus (HIV). The methods include contacting isolated mammalian CD4+ T cells with an effective amount of a thymic stromal derived lymphopoietin (TSLP) polypeptide or a therapeutically effective amount of nucleic acid encoding the TSLP polypeptide, thereby inducing proliferation of the T cells. Methods are also disclosed for treating an IgE mediated disorder, such as asthma. The methods include administering to a subject a therapeutically effective amount of a TSLP antagonist.

WIPO Patent Application No. WO/2005/007186 by Oft (2005) is based upon the discovery that the expression of the immune modulator, TSLP, is reduced during tumor progression and the addition of exogenous TSLP causes tumor regression. The application provides a method of modulating a neoplasm comprising contacting the neoplasm with an effective amount of TSLP or an agonist thereof. A method of diagnosing is described that comprises contacting a biological sample from a subject, with a TSLP or TLSPR antibody, under conditions suitable for the formation of an antibody:antigen complex, and detecting the complex by contacting a sample with an anti-TSLP or anti-TSLP-receptor.

With regard to OX40 Ligand (OX40L), U.S. Pat. No. 7,501,496, issued to Endl et al. (2009), is directed to anti-OX40L antibodies, and more particularly, to anti-OX40L antibodies and variants thereof that contain a Fc part derived from human origin and do not bind complement factor C1q. These antibodies are said to have new and inventive properties causing a benefit for a patient suffering from inflammatory diseases.

U.S. Patent Application Publication No. 2010/0098712, filed by Adler et al. (2010), is directed to pharmaceutical formulation of an antibody against OX40L. Briefly, the application is said to teach pharmaceutical formulations of an antibody against OX40L and processes for making the same. One such example is a pharmaceutical formulation comprising: 1 to 200 mg/mL of an antibody against OX40 ligand; 1 to 100 mM of a buffer; 0.001 to 1% of a surfactant; plus one of (a) 10 to 500 mM of a stabilizer, or (b) 10 to 500 mM of a stabilizer and 5 to 500 mM of a tonicity agent, or (c) 5 to 500 mM of a tonicity agent; at a pH in the range of from 4.0 to 7.0. The antibody is said to bind OX40L, contains a Fc part derived from human origin and not bind to complement factor C1q.

U.S. Patent Application Publication No. 2009/0053230, filed by Martin (2009) is directed to anti-OX40L antibodies and methods of using the same. The invention is said to provide anti-OX40L antibodies, and compositions comprising the same as well as methods of using these antibodies. One such example is a method for treating or preventing an immune disorder, the method comprising administering an effective amount of the anti-OX40L antibody to a subject in need of such treatment. Immune disorders are said to include an autoimmune disorder, including: asthma, atopic dermatitis, allergic rhinitis, inflammatory bowel disease, graft-versus-host disease, multiple sclerosis, or systemic lupus erythematosus.

### SUMMARY OF THE INVENTION

The present invention relates to compositions and methods involving the use of agents that neutralize or inhibit thymic stromal lymphopoietin (TSLP) and/or OX40L to inhibit tumor development and IL-13 secretion by human CD4+ T cells infiltrating breast cancer.

The instant invention in one embodiment relates to a therapeutic composition for the treatment of a tumor of epithelial origin in a human subject comprising one or more active agents that bind and neutralize the activity of a thymic stromal lymphopoietin (TSLP), wherein the one or more active agents are selected from the group consisting of an anti-TSLP antibody; an anti-TSLP antibody fragment; an anti-TSLP antibody-carrier conjugate; a TSLP binding fusion protein; a TSLP antagonist; a TSLP inhibitor a TSLP receptor antagonist; or a TSLP blocking agent optionally solubilized, dispersed or suspended in a suitable medium in an amount sufficient to inhibit development of the tumor. The composition of the instant invention is adapted to be administered intravenously, intramuscularly, subcutaneously, intraperitoneally or parenterally and reduces a tumor-induced inflammation, inhibits tumor development or both. In a specific aspect of the present invention the composition decreases a level of IL-4, IL-13 or both. In one aspect the tumor treated by the composition disclosed hereinabove is selected from a breast, prostate, kidney, lung or pancreatic cancer, wherein the active agent decreases the infiltration of Th2 cells into the tumor. In another aspect the composition further comprises one or more pharmaceutically acceptable excipients. In yet another aspect the composition further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

In another embodiment the present invention provides a method of treating or ameliorating symptoms of a cancer of epithelial origin in a human subject comprising the steps of: identifying the subject in need of treatment against the cancer and administering a therapeutically effective amount of a pharmaceutical composition sufficient to treat or ameliorate the symptoms of the cancer in the subject comprising one or more monoclonal or polyclonal antibodies selected from the group consisting of an anti-OX40L antibody, an anti-thymic stromal lymphopoietin (TSLP) antibody, or both.

In one aspect the composition further comprises at least one of an anti-IL-13, anti-IL-2, anti-IL-4, anti-TNFα, and receptors thereof. In another aspect the composition is administered intravenously; intramuscularly; subcutaneously; intraperitoneally; or parenterally. In another aspect the composition reduces a tumor induced inflammation, inhibits tumor development or both. In a related aspect the one or more antibodies are humanized. In another aspect the composition further comprises one or more pharmaceutically acceptable excipients. In yet another aspect the method described herein further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

Yet another embodiment of the present invention discloses a composition for treating a cancer of epithelial origin in a patient comprising a therapeutically effective amount of one or more active agents that neutralize the activity of a thymic stromal lymphopoietin (TSLP) in the patient in an amount sufficient to reduce the cancer. In one aspect of the composition described herein the one or more active agents are selected from the group consisting of an anti-TSLP antibody; an anti-TSLP antibody fragment; an anti-TSLP antibody-carrier conjugate; a TSLP binding fusion protein; a TSLP antagonist; a TSLP inhibitor; a TSLP receptor antagonist; or a TSLP blocking agent. In another aspect the anti-TSLP antibody is a humanized antibody. In another aspect the one or more active agents are dissolved, suspended or dispersed in suitable medium, wherein the one or more active agents that bind the TSLP decrease a level of an IL-4, IL-13 or both and reduces a tumor induced inflammation, inhibits tumor development or both. In another aspect the one or more active agents are adapted to be administered intravenously; intramuscularly; subcutaneously; intraperitoneally; or parenterally. In yet another aspect the cancer is selected from a breast; prostate; kidney; lung; or a pancreatic cancer. In one aspect wherein the active agent decreases the infiltration of Th2 cells into a tumor of the cancer. In another aspect the composition further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

One embodiment of the instant invention is related to a method of treating an individual who has a tumor, wherein the tumor secretes IL-13 comprising: administering to the individual, one or more agents that disrupt thymic stromal lymphopoietin (TSLP) activity selected from at least one of an anti-thymic stromal lymphopoietin (TSLP) antibody or antibody fragment; an anti TSLP protein; a TSLP antagonist; a TSLP inhibitor; a TSLP receptor antagonist; or TSLP blocking agent dispersed or solubilized in one or more optional pharmaceutically acceptable excipients. In specific aspects of the method the anti-TSLP antibody is a humanized antibody and the composition is administered orally; intravenously; intramuscularly; subcutaneously; intraperitoneally; or parenterally. In another aspect the composition reduces a tumor induced inflammation, inhibits tumor development or both and decreases a level of IL-4, IL-13 or both. In another aspect the tumor comprises a breast; a prostate; a kidney; a lung; or a pancreatic cancer. In yet another aspect the method further comprises administering at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

Another embodiment discloses a therapeutic composition for the treatment of a tumor of epithelial origin in a human subject comprising one or more active agents that bind and neutralize the activity of a OX40 Ligand, wherein the one or more active agents are selected from the group consisting of an anti-OX40 Ligand antibody; an anti-OX40 Ligand antibody fragment; an anti-OX40 Ligand antibody-carrier conjugate; a OX40 Ligand binding fusion protein; a OX40 Ligand antagonist; a OX40 Ligand inhibitor; an OX40 receptor antagonist; or a OX40 Ligand blocking agent optionally solubilized, dispersed or suspended in a suitable medium in an amount sufficient to reduce development of the tumor. In one aspect the composition is adapted to be administered intravenously; intramuscularly; subcutaneously; intraperitoneally; or parenterally. In another aspect the composition reduces a tumor-induced inflammation, inhibits tumor development or both by decreasing a level of IL-4, IL-13 or both. In yet another aspect the tumor is selected from a breast; prostate; kidney; lung; or pancreatic cancer.

In one aspect the active agent decreases the infiltration of Th2 cells into the tumor. In another aspect the composition further comprises one or more optional pharmaceutically acceptable excipients. In yet another aspect the composition further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

In yet another embodiment the instant invention is a method of treating an individual who has a tumor, wherein the tumor secretes IL-13 comprising: administering to the individual, one or more agents that disrupt OX40 Ligand selected from an anti-OX40 Ligand antibody; an anti-OX40 Ligand antibody fragment; an anti-OX40 Ligand antibody-carrier conjugate; a OX40 Ligand binding fusion protein; a OX40 Ligand antagonist; a OX40 Ligand inhibitor; or a OX40 Ligand blocking agent dispersed or solubilized in one or more optional pharmaceutically acceptable excipients. In one aspect of the method described hereinabove the antibody is a humanized antibody. In another aspect the one or more active agents are administered intravenously; intramuscularly; subcutaneously; intraperitoneally; or by any other suitable parenteral route. In yet another aspect the binding of the one or more active agents to the OX40 Ligand decrease a level of an IL-4, IL-13 or both. In related aspects of the method the tumor comprises a breast; a prostate; a kidney; a lung; or a pancreatic cancer and further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

In one embodiment the present invention discloses a composition for treating a cancer of epithelial origin in a patient comprising a therapeutically effective amount of one or more active agents that neutralize the activity of an OX 40 Ligand in the patient in an amount sufficient to reduce the cancer. In one aspect the one or more active agents an anti-OX40 Ligand antibody; an anti-OX40 Ligand antibody fragment; an anti-OX40 Ligand antibody-carrier conjugate; a OX40 Ligand binding fusion protein; a OX40 Ligand antagonist; a OX40 Ligand inhibitor; or a OX40 Ligand blocking agent dispersed or solubilized in one or more optional pharmaceutically acceptable excipients. In another aspect the antibodies are humanized. In another aspect the pharmaceutical composition treats the cancer by binding and neutralizing an OX40 Ligand. In another aspect the composition is administered orally; intravenously; intramuscularly; subcutaneously; intraperitoneally; or parenterally. In yet another aspect the composition reduces a tumor-induced inflammation, inhibits tumor development or both and the composition decreases a level of IL-4, IL-13 or both. In a specific aspect the tumor comprises a breast; a prostate; a kidney; a lung; or a pancreatic cancer. In another aspect the method further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

### BRIEF DESCRIPTION OF THE DRAWINGS

For a more complete understanding of the features and advantages of the present invention, reference is now made to the detailed description of the invention along with the accompanying figures and in which:

FIGS. 1A-1E show the inflammatory Th2 in breast cancer immune environment: FIG. 1A cytokine profiles as determined by Luminex in supernatants of tumor fragments upon 16 hrs PMA/Ionomycin activation. Ordinate: pg/ml, log scale; abscissa: indicated cytokines and the number of tissue samples from different patients tested, FIG. 1B cytokine profiles as determined by Luminex in supernatants of tumor fragments (T) and surrounding tissue (ST) from the same patient upon PMA/Ionomycin activation. Ordinate: pg/ml, log scale. Non-parametric paired rank test. n is number of patients analyzed, FIG. 1C shows the Spearman correlation between TNF-α (ordinate) and IL-13 (abscissa) secretion upon PMA/Ionomycin activation, FIG. 1D Polychromatic flow cytometry on single cell suspension analyzing the expression of cytokines by CD4+ T cells upon 5 hrs PMA/Ionomycin activation. Representative of four different patients from whom we have been able to obtain sufficient numbers of cells for 10 color analysis (Pt # 148, 155, 164 and 169), FIG. 1E shows immunofluorescence on frozen tissue sections, tissue from the same patient as in D. Triple staining with anti-CD3-FITC (green), anti-IL-13-Texas Red (red) and DAPI nuclear staining (blue);

FIGS. 2A-2G show the OX40L in breast cancer immune environment: (2A) OX40L ELISA in sonicate of 11 primary tumor tissue fragments; (2B) Immunofluorescence of primary tumor showing the presence of OX40L+ HLA-DR+ CD11c+ cells in tumor stroma. Representative of 57/60 tumors analyzed; (2C) Flow cytometry analysis of single cell suspension of primary breast cancer tumors and surrounding tissue; (2D-2E) mDCs are exposed to media or breast cancer cell line supernatant (2D) or sonicate of primary breast cancer tissue from patients (2E); (2F) summary of induction of OX40L+mDCs by sonicate of 28 primary tumor samples from patients; and (2G) Spearman correlation between the induction of OX40L+mDCs (ordinate) and IL-13 secretion (abscissa) from the same primary tumor tissue.

FIGS. 3A-3F show TSLP in breast cancer environment: (3A) Luminex analysis for TSLP in supernatants of breast cancer cell line Hs578T after 24 hrs of culture in the presence of PMA/Ionomycin. (3B) TSLP levels in sonicated primary breast tumors from 44 patients tested by Luminex, (3C) Expression of TSLP (red) by breast cancer cells MDA-MB 231 in vivo in subcutaneous tumors developed in immunodeficient mice. Actively dividing cells (Ki67+, green) were also positive for TSLP. (3D & 3E) expression of TSLP in primary tumors was found in 35 of 38 patients analyzed. TSLP+ cells were colocalized with IL-13 and cytokeratin 19. (3F) TSLP was also found tumor metastasis in lung and kidney in humanized mice.

FIGS. 4A to 4D show the blocking OX40L in vitro: mDCs exposed for 48 hrs to (4A) TSLP or (4B) supernatants of breast cancer cells Hs578T were co-cultured with allogenic naïve CD4+ T cells in the presence of 40 μg/ml of anti-OX40L or isotype control antibody. After one week cells were collected and re-stimulated for 5 hrs with PMA/Ionomycin for intracellular cytokines analysis, representative of 4 experiments, (4C) The effect of blocking OX40L was also observed with mDCs activated with soluble factors from human primary breast cancer tumors, one representative experiment of three patients tested. (4D) Summary of the effect of blocking OX40L during T cell stimulation by tumor activated DCs, graph shows the proportion of IL-13 secreting cells induced by dendritic cells activated with supernatants from breast cancer cell line Hs578T (left) or from primary breast cancer tumors (right).

FIGS. 5A-5E show the blocking TSLP in vitro: mDCs activated with soluble factors derived from breast cancer cell line Hs578T in the presence of 20 ug/ml of anti-TSLP antibody had not expression of OX40L (5A), and a low capacity to induce IL-13 secreting cells when were co-cultured with naïve CD4+ T cells in comparison with mDCs that were activated in the presence of isotype control antibody (5B). Histogram shows OX40L expression after 48 hrs activation, and dot-plots for intracellular cytokine staining after 5 hrs of re-stimulation with PMA/Ionomycin, representative of 3 experiments. (5C) Blockade with anti-TSLP antibody during activation of mDCs with soluble tumor factors from sonicated human breast cancer tumors, abolished the induction of OX40L expression in two (T60 and T97) of three tested patients. (5D) Effect of TSLP blockade was also observed in the induction of T cell cytokines using DCs activated with soluble tumor factors from sonicated human breast cancer tumors. TSLP blockade targets mainly triple positive cells for IFN-γ, IL-13 and TNF-α. Representative of three patients tested. Dot-plots show the profile of TNF-α and IFN-γ producing T cell. Blue dots represent IL-13+ T cells gated in the same sample analyzed by polychromatic flow cytometry. (5E) Analysis of three different experiments of the effect of TSLP blockage during DCs activation, graph shows the proportion of IL-13 secreting cells induced by dendritic cells activated with supernatants from breast cancer cell line Hs578T or from primary breast cancer tumors with anti-TSLP or isotype control antibody treatment.

FIGS. 6A-6C show the blocking OX40L-TSLP in vivo: (6A) mDCs were treated with anti-TSLP-R, media or control antibody during activation with TSLP. Then co-cultured with allogenic naïve CD4 T cells, after one week cells were collected and re-stimulated for intracellular cytokines analysis. Only mDCs treated with anti-TSLP-R antibody had a diminished capacity to induce IL-13 secreting T cells, indicating that the anti-TSLP-R antibody can block the effect of TSLP on mDCS. (6B) Blocking TSLP-R in mDCs during activation with supernatant of three different breast cancer cell lines (Hs578T, MDA-MB231 and MCF7) resulted in a low induction of IL-13 secreting T cells. (6C) Analysis of different experiments showing the effect of blocking TSLP-R in the induction of IL-13 secreting cells as described in (6B).

FIGS. 7A to 7F. Blocking OX40L-TSLP in vivo. (7A) study schema, humanized mice bearing tumor from Hs578T cells were transplanted with autologous total T cells and treated with 200 μg per injection of blocking anti-OX40L or isotype control antibody. T cells transplantation and antibody treatment were at day 3, 6, 9 after tumor cells implantation. Control group for T cell effects in tumor development were tumor-bearing humanized mice treated with PBS without T cells (gray line). Anti-OX40L treatment decrease tumor size (red line) in comparison with isotype control antibody treatment (green line). Average values from three experiments. (7B) Blockade of OX40L and cytokine profile of TILs obtained after 14 days of tumor implantation. (7C) Blockade of TSLP, tumor-bearing humanized mice transplanted with autologous T cells were treated with 200 μg per injection of neutralizing anti-TSLP antibody (red line), isotype control antibody (green line) or PBS (blue line). Control tumor-bearing mice without T cells (gray line). Representative of two experiments. (7D) Photographs of tumors removed from corresponding mice. (7E) Cytokine secretion in single cell suspensions from tumor upon overnight restimulation with PMA/Ionomycin. (7F) Blockade of TSLP, OX40L and IL-13 in the same cohort of mice.

FIG. 8 is a schematic showing the TSLP-OX40L-IL13 driven inflammation in breast cancer. In breast cancer tumor microenvironment, soluble factor TSLP expressed by breast cancer cells could induce mDCs activation through OX40L expression on the surface of mDCs. The activated OX40L+mDCs are able to further polarize Th2 cells, which produce IL-13 to contribute to the local inflammatory responses, and facilitate tumor development.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “cancer cells” refers to any cells that exhibit uncontrolled growth in a tissue or organ of a multicellular organism. The term “breast cancer” is understood to mean any cancer or cancerous lesion associated with breast tissue or breast tissue cells and can include precursors to breast cancer, for example, atypical ductal hyperplasia or non-atypical hyperplasia. The term “tumor” refers to an abnormal benign or malignant mass of tissue that is not inflammatory and possesses no physiological function.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless. The term “polypeptide” is a polymer of amino acid residues joined by peptide bonds, whether produced naturally or synthetically. Polypeptides of less than about 10 amino acid residues are commonly referred to as “peptides.”

The term “antibody” includes, but is not limited to, both naturally occurring and non-naturally occurring antibodies that are isolated and/or purified. Specifically, the term “antibody” includes polyclonal and monoclonal antibodies, and binding fragments thereof that continue to bind to antigen. Furthermore, the term “antibody” includes chimeric antibodies and wholly synthetic antibodies, and fragments thereof. Polyclonal antibodies are derived from the sera of animals immunized with the antigen. Monoclonal antibodies can be prepared using hybridoma technology (Kohler et al. Nature 256:495 (1975); Hammerling et al. in Monoclonal Antibodies and T-Cell Hybridomas, Elsevier, N.Y. pp. 563-681 (1981)). Antibodies also include includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in, e.g., WIPO Publication WO 98/24893, relevant portions incorporated herein by reference.

The term “humanized antibodies” refers to chimeric antibodies that comprise constant regions from human antibodies and hybrid variable regions in which most or all of the framework sequences are from a human variable region and all or most of the CDRs are from a non-human variable region. Humanized antibodies are also referred to as chimeric or veneered antibodies and are produced by recombinant techniques and readily available starting materials. Such techniques are described, for example, in UK Patent Application No. GB 2,188,638 A, relevant portions incorporated herein by reference.

The term “xenograft” as used throughout in this specification is synonymous with the term “heterograft” and refers to a graft transferred from an animal of one species to one of another species. Stedman's Medical Dictionary (Williams & Wilkins, Baltimore, Md., 1995).

The term “Th2” refers to a subclass of T helper cells that produce cytokines, such as IL-4, IL-5, IL-13, and IL-10, which are associated with an immunoglobulin (humoral) response to an immune challenge.

The term “inflammatory Th2” refers to a subclass of T helper cells that produce IL-4, IL-5, IL-13, and TNFα, and which elicit inflammatory reactions associated with a cellular, i.e. non-immunoglobulin, response to a challenge and which are associated with an immunoglobulin (humoral) response to an immune challenge.

The term “immunotherapy” refers to a treatment regimen based on activation of a pathogen-specific immune response an anti-tumor vaccine as described herein is a form of immunotherapy.

The term “vaccine composition” refers to a composition that can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes or both.

The term “immunofluorescence based techniques” or “immunocytochemical based techniques” encompasses various forms of such assays, as are known in the art. For example, and not by way of limitation, an immunofluorescence-based technique can use an unlabelled primary antibody and a fluorescently labeled secondary antibody (as illustrated, for example, in Example 1); or can use a primary antibody that carries a fluorescent tag to detect the phosphorylated H2AX molecule directly; or the primary antibody can carry a biotin molecule while the secondary antibody can carry both an avidin molecule (which binds specifically to biotin) and a fluorescence molecule. In the biotin/avidin approach, the binding of the secondary antibody is based on binding of biotin by avidin rather than the binding of an antibody of one species directed against a protein of another species. Other variations of such techniques that would be known to the skilled artisan as “immunofluorescence-based techniques” or “immunocytochemical-based techniques” can be used according to the invention. Likewise, detection can be made using analogous methods that utilize a modality other than fluorescence, such as chromogenic or colorimetric assays, radiologic assays, and so forth. Techniques such as immunocytochemical-based techniques can be used in conjunction with methods for counting cells, sorting cells, or other method for further characterizing cells. Exemplary methods include, but are not limited to, flow cytometry, laser scanning cytometry, fluorescence image analysis, chromogenic product imaging, fluorescence microscopy or transmission microscopy.

As used herein, the term “flow cytometry” refers to an assay in which the proportion of a material (e.g., ubiquitinated sperm) in a sample is determined by labelling the material (e.g., by binding a labelled antibody to the material), causing a fluid stream containing the material to pass through a beam of light, separating the light emitted from the sample into constituent wavelengths by a series of filters and mirrors, and detecting the light.

The terms “antagonist” or “inhibitor”, as used herein, refers to a molecule which, when bound to TSLP, IL-13 or OX40L, blocks or modulates the biological or immunological activity of the TSLP, IL-13 or the OX40L. Antagonists and inhibitors may include proteins, nucleic acids, carbohydrates, or any other molecules that bind to TSLP, IL-13, or OX40L.

The term “cytokine” as used herein includes any secreted polypeptide that affects the functions of other cells, and is a molecule that modulates interactions between cells in the immune or inflammatory response. A cytokine includes, but is not limited to monokines and lymphokines regardless of which cells produce them. For instance, a monokine is generally referred to as being produced and secreted by a mononuclear cell, such as a macrophage and/or monocyte but many other cells produce monokines, such as natural killer cells, fibroblasts, ip basophils, neutrophils, endothelial cells, brain astrocytes, bone marrow stromal cells, epideral keratinocytes, and B-lymphocytes. Lymphokines are generally referred to as being produced by lymphocyte cells. Examples of cytokines include, but are not limited to, interleukin-1 (IL-1), tumor necrosis factor-alpha (TNFα) and tumor necrosis factor beta (TNFβ).

The term “pharmaceutically acceptable” includes the carrier, diluent or excipient must be compatible with the other ingredients of the formulation and not deleterious to the recipient thereof.

The terms “administration of” or “administering a” as used herein refers to providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” refers to the amount of the subject compound that will elicit the biological or medical response of a tissue, system, animal or human that is being sought by the researcher, veterinarian, medical doctor or other clinician.

As used herein, the term “treatment” or “treating” refers to administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the term “in vivo” refers to being inside the body. The term “in vitro” used as used in the present application is to be understood as indicating an operation carried out in a non-living system.

As used herein, the term “chemotherapeutic” anti-cancer agents are those agents that reduce or eliminate cancer cells and may include, e.g., alkylating/carbamylating agents; platinum derivatives; antimitotic agents; tubulin inhibitors; topoisomerase inhibitors; nucleotide or nucleoside antagonists such as pyrimidine or purine antagonists; and folic acid antagonists.

As used herein, the term “target-specific” anti-cancer agents include those that specifically target cancer cells and include, e.g., taxanes; kinase inhibitors; phosphatase inhibitors; proteasome inhibitors; histone deacetylase inhibitors; heat shock protein inhibitors; vascular targeting agents (VAT); monoclonal antibodies (e.g., Trastuzumab; Rituximab; Alemtuzumab; Tositumomab; Cetuxcimab; Bevacizumab), as well as mutants; fragments and conjugates of monoclonal antibodies (e.g., Gemtuzumab; ozogamicin; or Ibritumomab tiuxetan); oligonucleotide based therapeutics; Toll-like receptor agonists; protease inhibitors; anti-estrogens hormonal therapeutics; anti-androgens hormonal therapeutics; luteinizing-hormone releasing hormone (LHRH) agents (e.g.; Leuprorelin; Goserelin; Triptorelin); aromatase inhibitors; bleomycin; retinoids; DNA methyltransferase inhibitors; alanosine; cytokines; interferons; and death receptor agonists. In one example, the tumor can be a breast cancer and the agent directed to the breast cancer.

Non-limiting examples of anti-cancer agents that may be useful in a combination therapy according to the present invention include, e.g., Actinomycin D; Abarelix; Abciximab; Aclarubicin; Adapalene; Alemtuzumab; Altretamine; Aminoglutethimide; Amiprilose; Amrubicin; Anastrozole; Ancitabine; Artemisinin; Azathioprine; Basiliximab; Bendamustine; Bevacizumab; Bexxar; Bicalutamide; Bleomycin; Bortezomib; Broxuridine; Busulfan; Campath; Capecitabine; Carboplatin; Carboquone; Carmustine; Cetrorelix; Chloram-Bucil; Chlormethine; Cisplatin; Cladribine; Clomifene; Cyclophosphamide; Dacarbazine; Daclizumab; Dactinomycin; Daunorubicin; Decitabine; Deslorelin; Dexrazoxane; Docetaxel; Doxifluridine; Doxorubicin; Droloxifene; Drostanolone; Edelfosine; Eflornithine; Emitefur; Epirubicin; Epitiostanol; Eptaplatin; Erbitux; Erlotinib; Estramustine; Etoposide; Exemestane; Fadrozole; Finasteride; Floxuridine; Flucytosine; Fludarabine; Fluorouracil; Flutamide; Formestane; Foscarnet; Fosfestrol; Fotemustine; Fulvestrant; Gefitinib; Genasense; Gemcitabine; Glivec; Goserelin; Gusperimus; Herceptin; Idarubicin; Idoxuridine; Ifosfamide; Imatinib; Improsulfan; Infliximab; Irinotecan; Ixabepilone; Lanreotide; Letrozole; Leuprorelin; Lobaplatin; Lomustine; Luprolide; Melphalan; Mercaptopurine; Methotrexate; Meturedepa; Miboplatin; Mifepristone; Miltefosine; Mirimostim; Mitoguazone; Mitolactol; Mitomycin; Mitoxantrone; Mizoribine; Motexafin; Mylotarg; Nartograstim; Nebazumab; Nedaplatin; Nilutamide; Nimustine; Octreotide; Ormeloxifene; Oxaliplatin; Paclitaxel; Palivizumab; Patupilone; Pegaspargase; Pegfilgrastim; Pemetrexed; Pentetreotide; Pentostatin; Perfosfamide; Piposulfan; Pirarubicin; Plicamycin; Prednimustine; Procarbazine; Propagermanium; Prospidium Chloride; Raloxifen; Raltitrexed; Ranimustine; Ranpirnase; Rasburicase; Razoxane; Rituximab; Rifampicin; Ritrosulfan; Romurtide; Ruboxistaurin; Sargramostim; Satraplatin; Sirolimus; Sobuzoxane; Sorafenib; Spiromustine; Streptozocin; Sunitinib; Tamoxifen; Tasonermin; Tegafur; Temoporfin; Temozolomide; Teniposide; Testolactone; Thiotepa; Thymalfasin; Tiamiprine; Topotecan; Toremifene; Trail; Trastuzumab; Treosulfan; Triaziquone; Trimetrexate; Triptorelin; Trofosfamide; Uredepa; Valrubicin; Vatalanib; Verteporfin; Vinblastine; Vincristine; Vindesine; Vinorelbine; Vorozole; and Zevalin. The person skilled in the art is aware on the base of his/her expert knowledge of the total daily dosage(s) and administration form(s) of the additional therapeutic agent(s) co-administered with the active agents of the present invention and in the methods taught herein. The total daily dosage(s) can vary within a wide range.

While practicing the present invention, the various compounds according to the present invention (anti-TSLP agents and/or anti-OX40L) may be administered in combination therapy separately, sequentially, simultaneously, concurrently or chronologically staggered (such as e.g. as combined unit dosage forms, as separate unit dosage forms, as adjacent discrete unit dosage forms, as fixed or non-fixed combinations, or as admixtures, each of which may be provided alone or as part of a kit) with one or more standard therapeutics, e.g., one or more of the anti-cancer agents discussed hereinabove above.

When used alone or in combination with an anti-cancer agent, the active agents of the present invention may be provided, separately, sequentially, simultaneously, concurrently or chronologically staggered.

The present invention discloses antibodies neutralizing thymic stromal lymphopoietin (TSLP) and/or OX40L to inhibit tumor development and IL-13 secretion in a xenograft model of human breast cancer. It has been found that the TSLP-OX40L-IL-13 axis contributes to breast cancer pathogenesis. The TSLP-neutralizing antibodies of the instant invention block the upregulation of OX40L by DCs exposed to breast cancer, thereby blocking their capacity to generate inflammatory Th2 cells.

The present inventors use primary breast cancer tumor samples and a xenograft model of human breast cancer to analyze how breast cancer can modulate the immune system to facilitate tumor development. The inventors demonstrate that breast cancer cells can produce TSLP, a cytokine known to induce dendritic cells (DCs) to express OX40 ligand (OX40L). Indeed, DCs exposed to breast cancer ex vivo acquire OX40L expression and OX40L+DCs can be found in primary breast cancer tumor infiltrates. These DCs drive IL-13⁺ TNFα⁺IL-10^{neg}CD4⁺ T cells (inflammatory Th2).

The present invention addresses the need for developing novel therapeutic approaches to improve the survival of patients with breast cancer by Immunotherapy. Breast tumors are infiltrated with Th2 cells that help breast tumor development. Thus, one therapeutic approach could be to block the generation and action of these tumor promoting effector CD4⁺ T cells secreting type 2 cytokines. The present invention demonstrates that breast cancer shows inflammatory and allergic-like environment driven by bioavailability and activity of TSLP which induces and maintains pro-tumor CD4⁺ T cells via OX40L-expressing dendritic cells. Thus, TSLP, and/or down-stream pathways, represent novel potential therapeutic targets.

Despite declining mortality rates, breast cancer ranks second among cancer related deaths in women. Worldwide, it is estimated that more than one million women are diagnosed with breast cancer every year, and more than 410,000 will die from the disease (Coughlin and Ekwueme, 2009). Immunotherapy could represent an attractive option for treatment-resistant breast cancer (Jones and Buzdar, 2009) (Anderson, 2009; Finn, 2008; Finn et al. 1995; Jaini et al. 2010). Indeed, the last decade brought about the demonstration that breast cancer is immunogenic (Disis et al. 1994; Disis et al. 2009). Perhaps the most compelling evidence for effective anti-tumor immunity in breast cancer comes from studies on paraneoplastic diseases (Darnell, 1994). Onconeural antigens, which are normally expressed on neurons, the immune privileged sites, are also expressed in some cases of breast cancer (Darnell, 1996). In these patients, a strong antigen-specific CD8⁺ T cell response is generated, which provides effective tumor control but also an autoreactive neurologic disease, paraneoplastic cerebellar degeneration (Albert et al. 1998). Nevertheless, in the majority of cases, the natural immunity to breast cancer is not protective, highlighting the need to develop strategies to boost immune resistance to cancer.

One approach to boosting patient resistance could be vaccination. Indeed, cancer vaccines are in a renaissance era prompted by recent phase III clinical trials showing clinical benefit to the patients. Vaccines act through dendritic cells (DCs) that induce, regulate and maintain T cell immunity. Critical to the design of improved vaccines is the demonstration that DCs are composed of distinct subsets (Caux et al. 1997; Dudziak et al. 2007; Klechevsky et al. 2008; Luft et al. 2002; Maldonado-Lopez et al. 1999; Pulendran et al. 1999) which respond differentially to distinct activation signals, (functional plasticity) (Steinman and Banchereau, 2007), both contributing to the generation of unique adaptive immune responses. Thus, in the steady state, non-activated (immature) DCs present self-antigens to T cells, which leads to tolerance (Hawiger et al. 2001; Steinman et al. 2003). Once activated (mature), antigen-loaded DCs are geared towards the launching of antigen-specific immunity (Brimnes et al. 2003; Finkelman et al. 1996) leading to the proliferation of T cells and their differentiation into helper and effector cells. The two major subsets are the myeloid DCs (mDCs) and the plasmacytoid DCs (pDCs). pDCs are considered as the front line in anti-viral immunity owing to their capacity to rapidly produce high amounts of type I interferon (Liu, 2005; Siegal et al. 1999). The best studied human mDC subsets in the tissue are those from skin, where three subsets can be identified. The epidermis hosts only Langerhans Cells (LCs) while the dermis displays two mDC subsets, CD1a⁺ DCs and CD14⁺ DCs, as well as macrophages (Klechevsky et al. 2008; Merad et al. 2008; Nestle et al. 2009; Zaba et al. 2007). CD14⁺ dermal DCs specialize in generation of humoral immunity with IL-12 being a major cytokine (Caux et al. 1997) (Klechevsky et al. 2008), whereas LCs specialize in the priming of high avidity antigen-specific CD8⁺ T cells (Klechevsky et al. 2008). mDCs can be further polarized by other cells and their products. For example, IL-10 polarized-mDCs generate anergic CD8+ T cells that are unable to lyse tumors (Steinbrink et al. 1999) as well as CD4+ T cells with regulatory/suppressor function (Levings et al. 2005). In contrast, thymic stromal lymphopoietin (TSLP)-polarized mDCs are conditioned to expand T cells producing type 2 cytokines (Gilliet et al. 2003; Soumelis et al. 2002).

DCs can be generated ex-vivo from bone marrow progenitors or blood precursors and loaded with selected antigens for injection to patients. In another approach, DCs can be specifically targeted in vivo with anti-DC antibodies decorated with antigens. This however requires understanding how DCs are affected by the tumor environment.

Another approach to breast cancer immunotherapy could be to block the generation and action of tumor promoting effector T cells secreting type 2 cytokines. Indeed, a number of studies in murine models of cancer have demonstrated that type 2 cytokines are involved in tumorigenesis. For example, IL-13 produced by NKT cells induces myeloid cells to make TGF-β which ultimately inhibits CTL functions (Berzofsky and Terabe, 2008). Spontaneous autochthonous breast carcinomas arising in Her-2/neu transgenic mice appear more quickly when the mice are depleted of T cells, evidence for T-cell mediated immunosurveillance slowing tumor growth. This immunosurveillance could be further enhanced by blockade of IL-13, which slowed the appearance of these autologous tumors compared to control antibody-treated mice. A spontaneous mouse breast cancer model recently highlighted the role of Th2 cells, which facilitate the development of lung metastasis through macrophage activation (DeNardo et al. 2009).

The present inventors have previously reported that breast cancer tumor beds are always infiltrated with immature DCs. In contrast, peri-tumoral areas are infiltrated with mature DCs in ˜60% of cases (Bell et al. 1999). (15;87) The tumor cells polarize DCs into a state that drives the differentiation of naïve CD4′ T cells into IL-13-secreting T cells (Aspord et al. 2007). These Type 2 T cells in turn facilitate breast cancer tumor development as shown in xenograft model where it can be partly inhibited by administration of IL-13 antagonists. The present invention show that mDCs respond to breast cancer-derived TSLP by increased expression of OX40L leading to the generation of inflammatory Th2 cells that promote tumor development.

Isolation and culture of myeloid dendritic cells: DCs were purified from buffy coat of blood from healthy donors. Briefly, DCs were enriched from mononuclear cells by negative selection using a mixture of antibodies against linage markers for CD3, CD14, CD16, CD19, CD56 and glycophorin A (Dynabeads® Human DC Enrichment Kit, Invitrogen). Cells from negative fraction were immuno-labeled with anti-human FITC-labeled linage cocktail (CD3, CD14, CD16, CD19, CD20 and CD56, BD biosciences Cat. 340546); PE-labeled CD123 (mIgG1, clone 9F5, BD biosciences Cat.340545), QR-labeled HLA-DR (mIgG2a, clone HK14, Sigma-Aldrich Cat. R8144) and APC-labeled CD11c (mIgG2b, clone S-HCL-3, BD biosciences Cat. 340544). DCs (lin⁻, CD123⁻, HLA-DR⁺, CD11c⁺) were sorted in a FACS Aria cytometer (BD Bioscience). DCs were seeded at 100×10³ cells/well in 200 μl of medium (RPMI supplemented with glutamine 2 mM, penicillin 50 U/ml, streptomycin 50 μg/ml, MEM non-essential amino acids 0.1 mM, HEPES buffer 10 mM, sodium pyruvate 0.1 mM and 10% of human AB serum). DCs were cultured with medium alone or in the presence of 20 ng/ml of TSLP, or different tumor derived products. After 48 hrs DCs were harvested and washed. The stimulated cells were stained for phenotype analysis or co-culture with allogeneic naïve CD4 T cells.

Immuno-fluorescence: Frozen sections (6 μm) from tissues were fixed with cold acetone for 5 minutes. The sections were labeled with 5 μg/ml of anti-OX40L antibody (mouse IgG1, 8F4), following by anti-mouse IgG conjugated to Texas-Red (Jackson Immunoresearch, West Grove, Pa.). For IL-13 tissue was labeled with 10 μg/ml of anti-IL-13 (polyclonal goat IgG, AF-123-NA, R & D System Inc) following with Texas red anti-goat IgG (Jackson Immunoresearch, West Grove, Pa.). TSLP was detected with 10 μg/ml of mouse anti-TSLP antibody prepared in-house (mIgG1, clone 14C3.2E11). Cytokeratin 19 was labeled with monoclonal antibody clone A53-BA2 (IgG2a, abcam), following by Alexa Fluor 568 goat anti-mouse IgG2a (A-21134, Invitrogen). Direct labeled antibodies used were FITC anti-HLA-DR (mouse IgG2a, L243, BD biosciences Cat. 347363), FITC anti-CD11c (clone, KB 90, DAKO), FITC anti-Ki67 (clone ki-67, DAKO) and Alexa-Fluor 488 anti-CD3 (mIgG1, UCHT1, BD Bioscience Cat. 557694). Finally, sections were counterstained for 2 minutes with the nuclear stain DAPI (3 μM in PBS. Invitrogen, Molecular Probes Cat. D21490). To confirm specificity of TSLP staining, primary anti-TSLP antibody was pre-incubated with 100 μg of recombinant human TSLP (R and D system, Inc) for 30 minutes at RT, before stain tissue sections that previously showed to be TSLP positive.

Cytospin staining: mDCs were plate in 96-well flat bottom plates at 10⁵ mDCs/well. DCs were cultured for 48 hrs with medium alone or 40% of tumor supernatant from HS578T cells cultured in vitro. Then cells were harvested and spun on slides (cytospin) and stored at −20° C. For staining cytospin preparations were labeled with 1.25 μg/ml of anti-HLA-DR FITC (mIgG2a, clone L243, BD biosciences Cat. 347363) or Isotype control FITC. After that cells were counterstained for 2 minutes with the nuclear stain DAPI.

Flow Cytometry Analysis: Cell suspensions from human breast carcinoma tissue and tumor or draining lymph nodes from humanized mice were used for phenotypic characterization of leukocytes. Cell suspensions were obtained by digestion with 2.5 mg/ml of collagenase D (Roche Diagnostics, Indianapolis, Ind.), and 200 U/ml of DNAse I (Sigma-Aldrich, St. Louis, Mo.) for 30 to 60 minutes at 37° C. The anti-human antibodies used were FITC-labeled linage cocktail (CD3, CD14, CD16, CD19, CD20 and CD56, BD biosciences Cat. 340546); PE-labeled OX40L (mIgG1, clone Ik-1, BD biosciences Cat. 558164); QR-labeled HLA-DR (mIgG2a, clone HK14, Sigma-Aldrich Cat. R8144); APC-labeled CD11c (mIgG2b, clone S-HCL-3, BD biosciences Cat. 340544); PerCP-labeled CD3 (mIgG1, clone SK7, BD biosciences Cat. 347344); PECy7-labeled CD4 (mIgG1, clone SK3, BD biosciences Cat. 557852); APCCy7-labeled CD8 (mIgG1, clone SK1, BD biosciences Cat. 557834); FITC-labeled IL-4 (mIgG1, clone 3007, R & D Systems, Inc., Cat. IC204F); Pacific blue labeled IL-10 (rat IgG1, clone JES3-9D7, e-biosciences Cat. 57-7108-73); PE-labeled IL-13 (rat IgG1, clone JES10-5A2 BD biosciences Cat. 559328); APC-labeled TNF-α (mIgG1, clone 6401.1111, BD biosciences Cat. 340534); Alexa Fluor 700 labeled IFN-γ (mIgG1, clone B27, BD biosciences Cat. 557995). Cells were incubated with the antibodies for 30 minutes at 4° C. in the dark, then washed three times and fixed with 1% paraformaldehyde to be analyzed in a FACScalibur or LSR-II cytometer (Becton Dickinson). For intracellular cytokines, cells were stained using BD cytofix/cytoperm fixation/permeabilization kit according to the manufacturer directions.

Tumor factors preparation: Tumor factors were obtained from supernatant of HS578T cells cultured in vitro or by sonication from tumor cell lines, human breast tumor tissue or tumors from humanized mice. Briefly, cell lines were culture in medium (RPMI supplemented with glutamine 2 mM, penicillin 50 U/ml, streptomycin 50 μg/ml, MEM non-essential amino acids 0.1 mM, HEPES buffer 10 mM, sodium pyruvate 0.1 mM and 10% of fetal calf serum), and when the cells reached 90% of confluence fresh medium was added and left the cells in culture for additional 48 hrs. For sonication cells or tissues were placed in PBS and were disrupted during 30 seconds at 4° C., with the power output adjusted at 4.5 level of the 60 sonic dismembrator (Fisher Scientific). Cellular debris were removed by centrifugation and the supernatant was collected and stored at −80° C.

Cytokine analysis: Tumor samples from patients diagnosed with breast carcinoma (in situ and invasive duct and/or mucinous carcinoma of the breast, as well as lobular carcinoma) were obtained from the Baylor University Medical Center Tissue Bank. Tumors and draining lymph nodes from humanized mice implanted with breast cancer cell line H578T were also analyzed. Whole-tissue fragments (4×4×4 mm, 0.015-0.030 g, approximately), were placed in culture medium with 50 ng/ml of PMA (Sigma-Aldrich Cat. P8139), and 1 μg/ml of ionomycin (Sigma-Aldrich Cat. I0634) for 18 h. Cytokine production was analyzed in the culture supernatant by Cytokine Multiplex Assay. For intracellular staining, cells were resuspended at a concentration of 10⁶ cells/ml in medium and activated for 5 h with PMA and ionomycin, Brefeldin A (Golgiplug, BD biosciences Cat. 554725) and monensin (Golgistop BD biosciences Cat. 555029) were added for the last 2.5 h.

DC-T cell co-cultures: Total CD4+ T cells were enriched from PBMC of healthy donors using magnetic depletion of other leukocytes (EasySep® Human CD4′ T Cell Enrichment Kit, Stemcell technologies Cat. 19052). Naive CD4 T cells were sorted based on the expression of CD4+ CD27+ and CD45RA+. Activated mDCs with medium, TSLP or tumor derived factors were co-cultured with naïve CD4+ T cells in a ratio 1:5 during 7 days. Thereafter the cells were washed and re-stimulated 5 hrs with PMA (50 ng/ml) and Ionomycin (1 μl/ml), Brefeldin A and monensin were added for the last 2.5 hrs. Followed by surface and intracellular staining. For blocking OX40L, tumor activated mDCs were co-cultured with naïve CD4+ T cells in the presence of 50 μg of anti-OX40L (Ik-5 clone) or control IgG2a isotype antibody. For blocking TSLP tumor derived factors were pre-incubated with 20 μg/ml of anti-TSLP antibody (Rabbit, AB 19024) or normal rabbit IgG (R and D systems, Cat.AB-105-C) at RT for 30 minutes, previous to DC activation. Then DCs were activated with the neutralized tumor derived factors and finally co-cultured with naïve CD4+ T cells for 7 days. For TSLPR blocking, DCs were pre-incubated with anti-TSLP receptor antibody (PAB1708, clone AB81_{—}85.1F11, mouse IgG1) for 3 min at room temperature.

Tumor bearing humanized mice: CD34+ hematopoietic progenitor cells (HPCs) were obtained from apheresis of adult healthy volunteers mobilized with G-CSF and purified as previously described. The CD34− fraction of apheresis was Ficoll purified, and obtained PBMCs were stored frozen and used as a source of autologous T cells. Three million CD34+HPCs were transplanted intravenously into sublethally irradiated (12 cGy/g body weight of 137Cs γ irradiation) NOD/SCID/β2m−/− mice (Jackson ImmunoResearch Laboratories). After 4 weeks of engraftment 10 million Hs578T breast cancer cells were harvested from cultures and injected subcutaneously into the flanks of the mice. Mice were reconstituted with 10 million CD4+ T cells and 10 million CD8+ T cells autologous to the grafted CD34+HPCs. CD4+ and CD8+ T cells were positively selected from thawed PBMCs using magnetic selection according to the manufacturer's instructions (Miltenyi Biotec). The purity was routinely >90%. T cells were transferred at days 3, 6 and 9 post tumor implantation. For experiments with NOD/SCID/β2m−/− mice, they were sublethally irradiated the day before tumor implantation. Then mice were reconstituted with 1 million of monocyte derived DCs (MDDCs) and autologous T cells as described above. MDDCs were generated from the adherent fraction of PBMCs by culturing with 100 ng/ml GM-CSF (Berlex) and 10 ng/ml IL-4 (R&D Systems). Tumor size was monitored every 2-3 d. Tumor volume (ellipsoid) was calculated as follows: [(short diameter)²×long diameter]/2.

Blocking in vivo experiments: Tumor bearing humanized mice transferred with autologous T cells, were injected intra-tumor with 200 μg of blocking antibody anti-OX40L clone IK-5 or isotype control mIgG2a at days 3, 6 and 9 post tumor implantation. For blocking TSLP mice were injected intra-tumor with 100 μg of anti-TSLP antibody AB 19024 (rabbit IgG) or normal rabbit IgG at day 0, following by three injections of 200 μg of the antibodies at days 3, 6 and 9 post tumor implantation. For IL-13 blockage, 100 μg of neutralizing antibody prepared in house (mIgG1, clone 13G1.B2) or isotype control antibody were injected intra-tumor at days 3, 6 and 9 post tumor implantation.

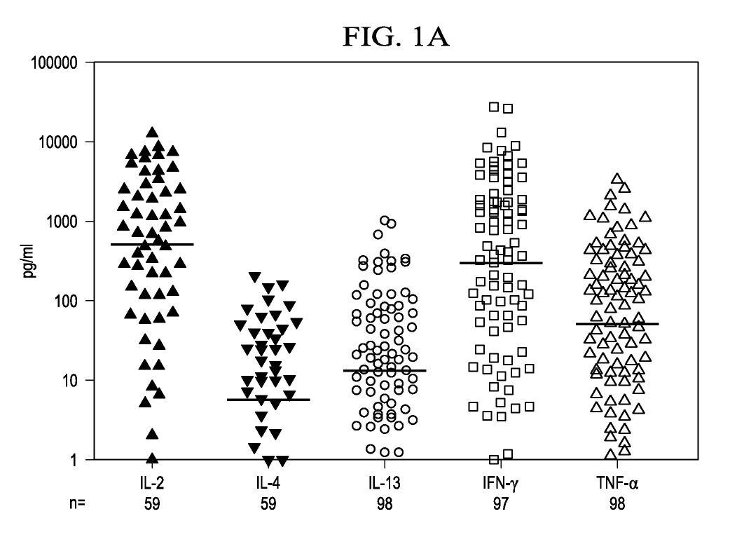

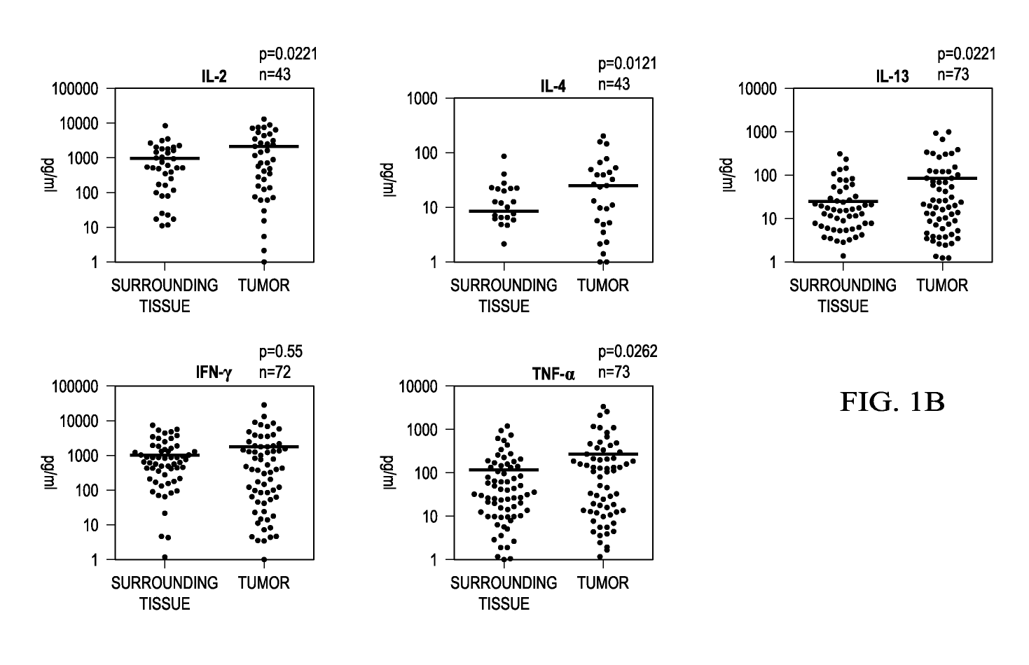

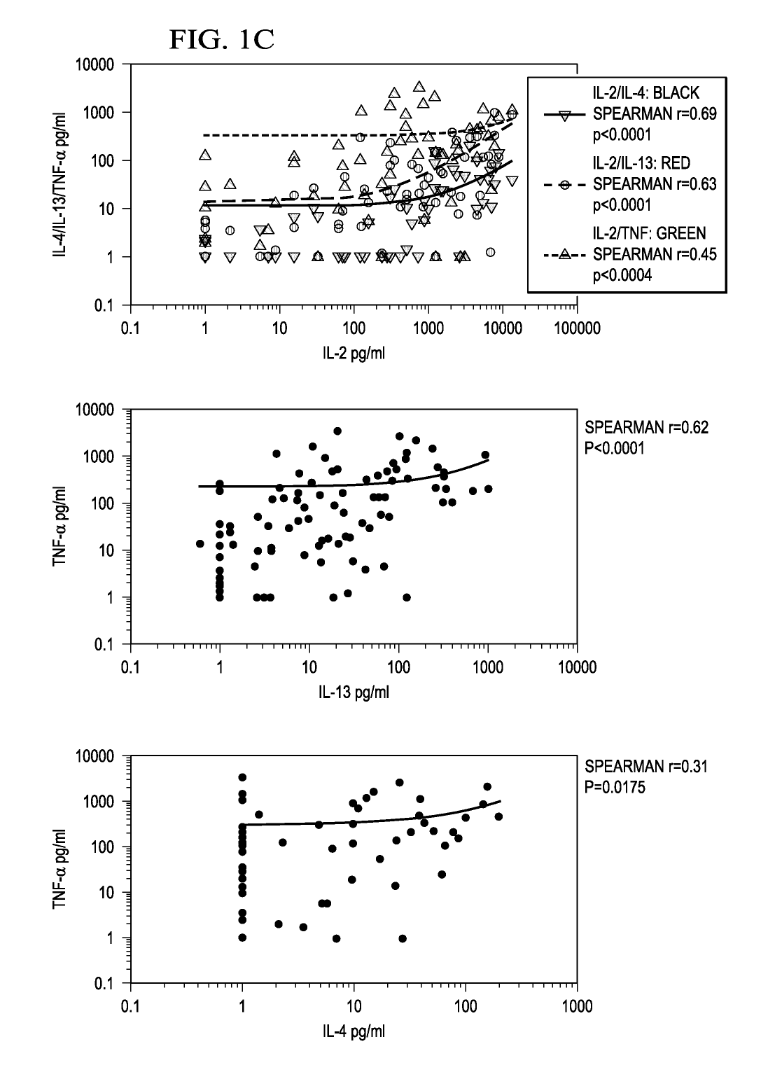

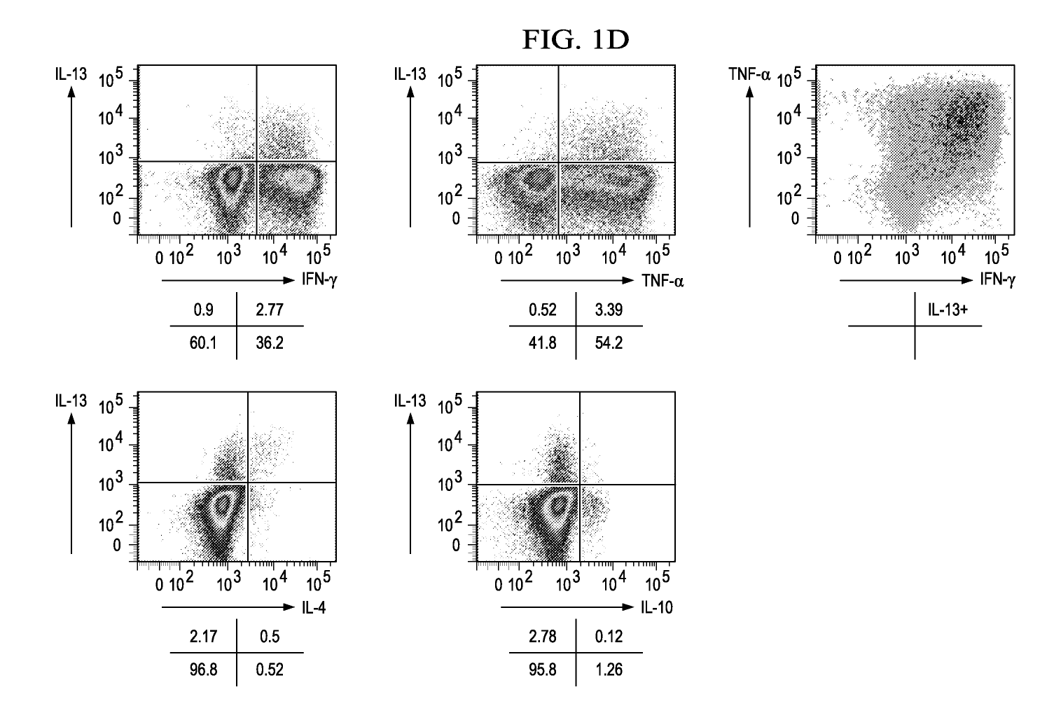

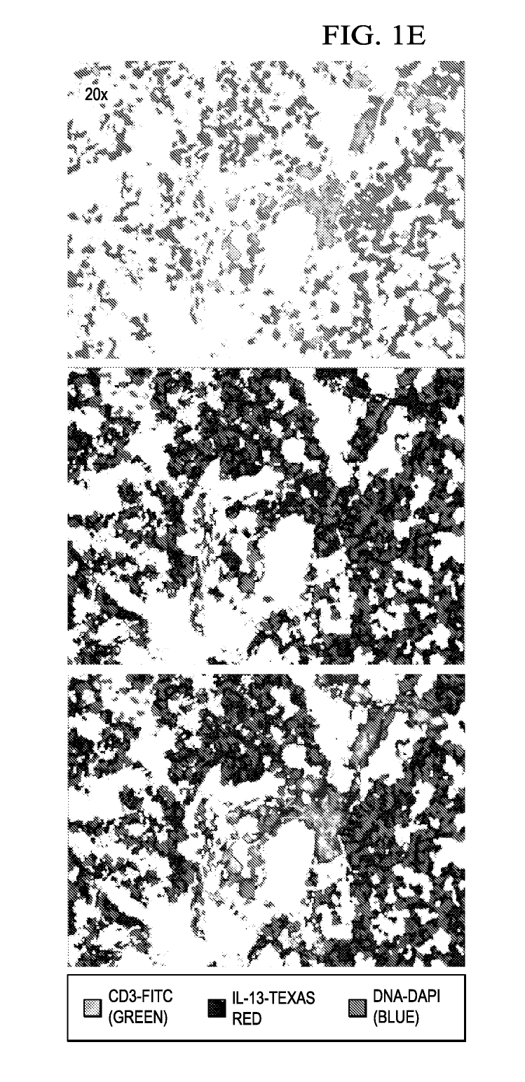

Inflammatory Th2 cells in primary breast cancer tumors: A previous study by the present inventors using a pilot cohort of 19 samples of primary breast cancer tumors revealed the secretion, upon activation with PMA/ionomycin, of both type 1 (IFN-γ) and type 2 (IL-4 and IL-13) cytokines (Aspord et al. 2007). The current study analyzes a total of 99 consecutive samples. Supernatants of activated tumor fragments display high levels of IFN, IL-2, IL-4, IL-13 and TNF (FIG. 1A and Table 1). Supernatants from tumor sites contained significantly higher levels of IL-2, type 2 (IL-4 and IL-13) and inflammatory (TNF-α) cytokines than those from macroscopically uninvolved surrounding tissue (FIG. 1B and Table 1). IFN-γ did not correlate with other cytokine levels. However, levels of IL-2 were correlated with other cytokines IL-4, IL-13 and TNF-α (n=59, FIG. 1C). Furthermore, TNF-α levels were correlated with those of IL-13 (p<0.0001, r=0.62, n=98) and IL-4 (p=0.0175, r=0.31, n=59) (FIG. 1C).

To identify the cells producing these cytokines, single-cell suspensions were prepared from tumors, activated for 5 hrs with PMA/ionomycin, stained with antibodies against T cells and cytokines and analyzed by flow cytometry. Gated viable CD4⁺ CD3⁺ T cells expressed IL-13 (3.67%), most of them co-expressing IFN-γ and TNF-α (FIG. 1D and supplement FIG. 1). A small fraction of IL-13⁺CD4⁺ T cells co-expressed IL-4 but none expressed IL-10 (FIG. 1D). Such T cells have been referred to as inflammatory Th2 cells that are involved in allergic inflammatory diseases (Liu et al. 2007). The analysis of frozen tissue sections further demonstrated that infiltrating T cells in primary breast cancer tumors express IL-13 (FIG. 1E). Thus, breast cancer tumors are infiltrated with inflammatory Th2 cells.

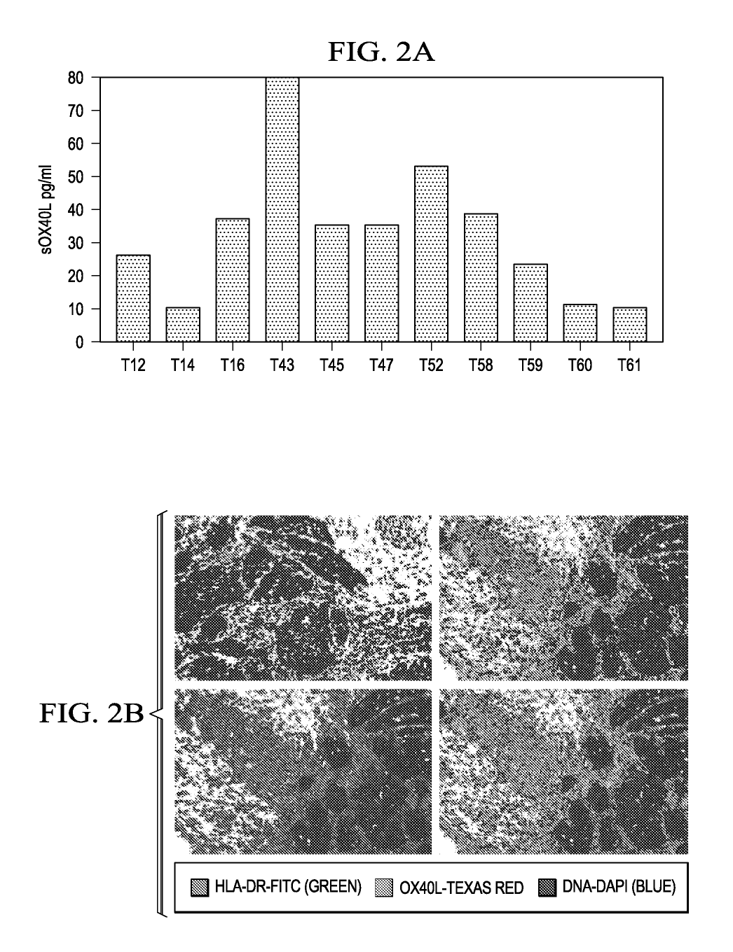

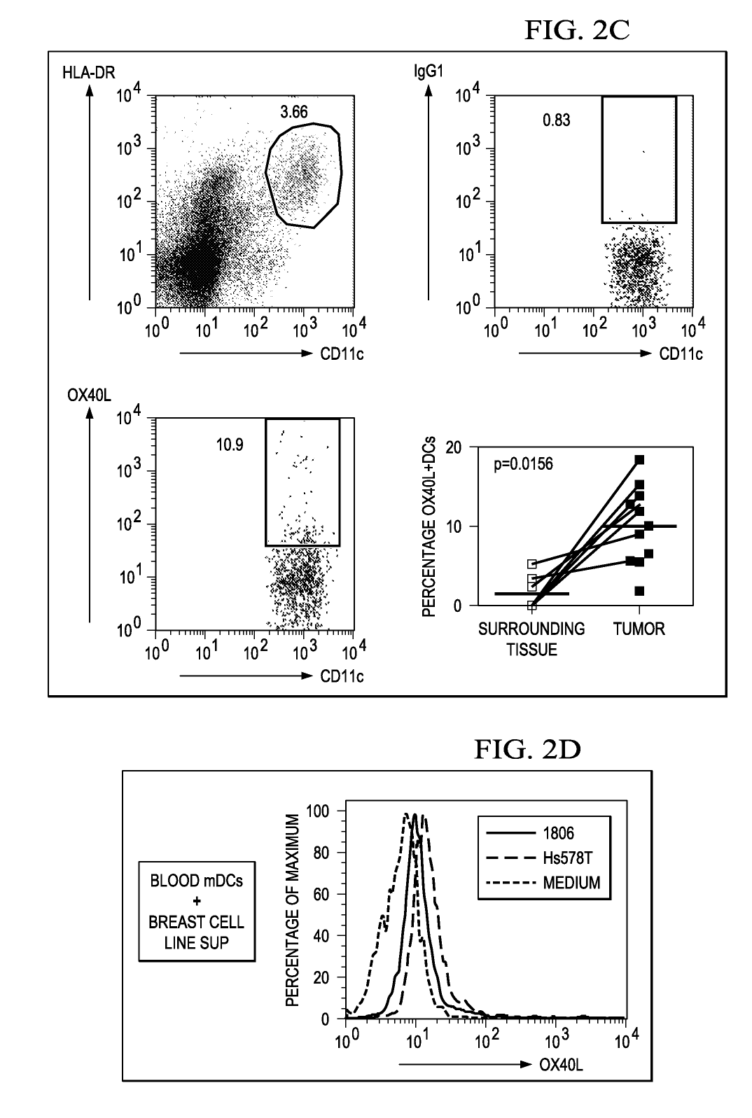

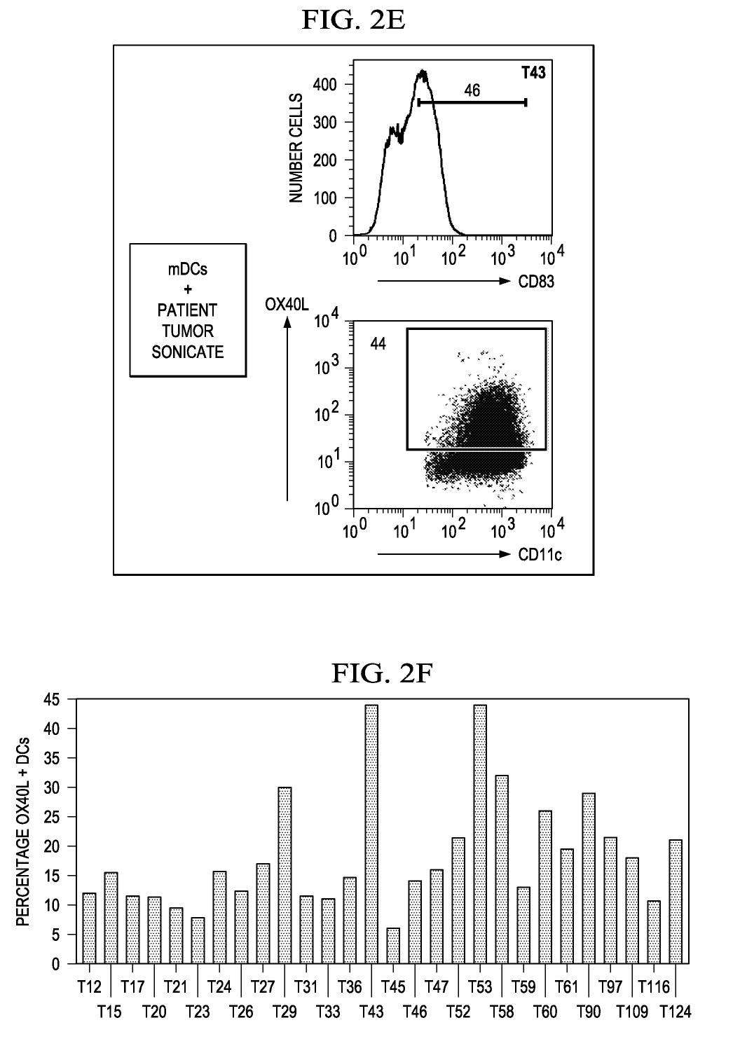

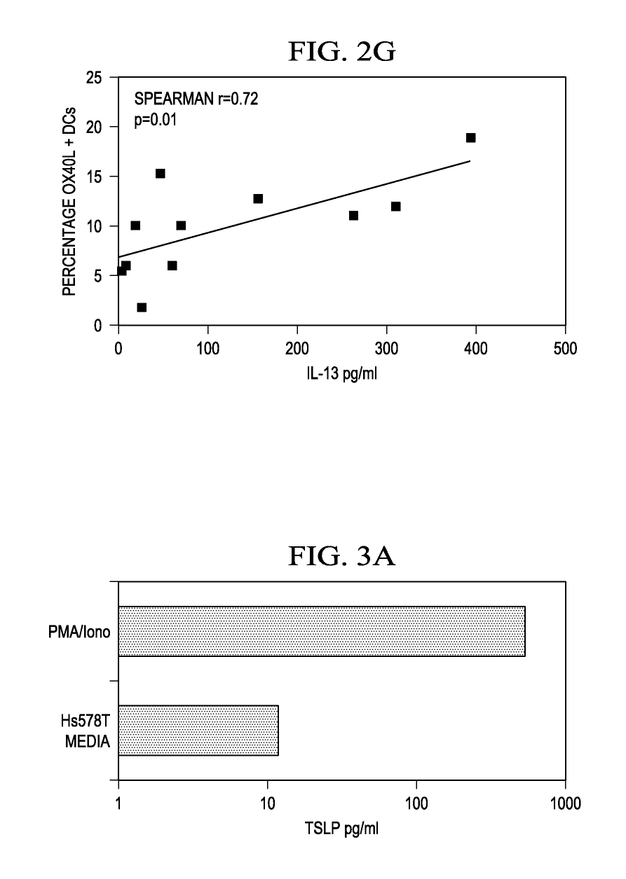

DCs infiltrating breast cancer tumors express OX40 ligand: Because OX40 ligation drives the differentiation of CD4′ T cells into inflammatory Th2 (Ito et al. 2005), we analyzed the presence of OX40L in primary breast cancer tumors. Soluble OX40L could actually be detected by ELISA in supernatants from sonicated breast cancer tumor fragments (FIG. 2A). Immunofluorescence staining of frozen tissue sections of primary breast cancer tumors showed the expression, in 57 out of 60 analyzed tumors, of OX40L by a majority of HLA-DR^{high} cells (FIG. 2B). These OX40⁺ cells are located in peri-tumoral areas (FIG. 2B). Flow cytometry analysis of single cell suspensions further confirmed the expression of OX40L by a fraction of HLA-DR^{high} CD11c^{high} mDCs (FIG. 2C). Paired analysis demonstrated that the tumor beds express higher percentages of OX40L⁺ mDCs than the surrounding tissue (p=0.0156, n=7 paired samples, mean±SE for surrounding tissue=1.5%±0.8, n=7; and for breast cancer tumors 11%±1.67, n=12, respectively; FIG. 2C). Thus, breast cancer tumors are infiltrated with OX40L⁺ mDCs.

Breast cancer tumors produce soluble factors that induce OX40L on DCs: To identify the breast cancer tumor factor(s) which induce(s) OX40L on mDCs, LIN^{neg}HLA-DR⁺ CD123⁻ CD11c⁺ mDCs were sorted from healthy volunteers blood and exposed to breast cancer supernatants. These were generated from: 1) established breast cancer cell lines expanded in vitro Hs578T, MDA-MD-231, MCF7, HCC-1806, and T47D (Table 2); and 2) breast cancer tumors established in vivo by implanting breast cancer cell lines in immunodeficient mice (Aspord et al. 2007). As illustrated in FIG. 2D, mDCs exposed for 48 hours to Hs578T and HCC-1806 supernatants expressed OX40L. Four of the five breast cancer cell lines, with the notable exception of T47D, induced OX40L expression on mDCs.

TABLE 1
Cell line characteristics.

| | Original Tissue | | | | | |
|---|---|---|---|---|---|---|
| Cell line | (derived from | Histopathological | Receptor status | | | |
| (reference) | metastatic site) | diagnosis | PR | ER | her2 | IGF-IR |
| Hs 578T | Breast | Primary, | − | − | low | low |
| | | Carcinosarcoma | | | | |
| HCC-1806 | Breast | Primary, | − | − | − | + |
| | | Acantholytic | | | | |
| | | Squamous cell | | | | |
| | | Carcinoma | | | | |
| MDA- | Breast (Pleural | Metastatic, | − | − | − | Low |
| MB-231 | effusion) | Invasive | | | | |
| | | Ductal Carcinoma | | | | |
| MCF7 | Breast (Pleural | Metastatic, | + | + | low | + |
| | effusion) | Invasive | | | | |
| | | Ductal Carcinoma | | | | |
| T47D | Breast (Pleural | Metastatic, Ductal | + | + | + | + |
| | effusion) | Carcinoma | | | | |

To determine whether primary breast cancer tumors could also regulate OX40L expression, fragments of primary tumors were sonicated, centrifuged, filtered and used in cultures with blood mDCs. As illustrated in FIG. 2E, mDCs acquired both CD83 (a DC maturation marker) and OX40L. The analysis of soluble components of 28 consecutive primary breast cancer tumor samples showed induction of mDCs maturation and OX40L expression in 6%-44% of mDCs (median=15.6%, mean±SE=18.4%±1.85%; FIG. 2F). The capacity of primary tumor soluble fraction to induce OX40L correlated positively with IL-13 secretion upon exposure to PMA/Ionomycin (p=0.01, r=0.72, n=11, FIG. 2G) suggesting a link between these two biological features. Thus, breast cancer cells produce (a) factor(s) that activate mDCs and induce them to express OX40L.

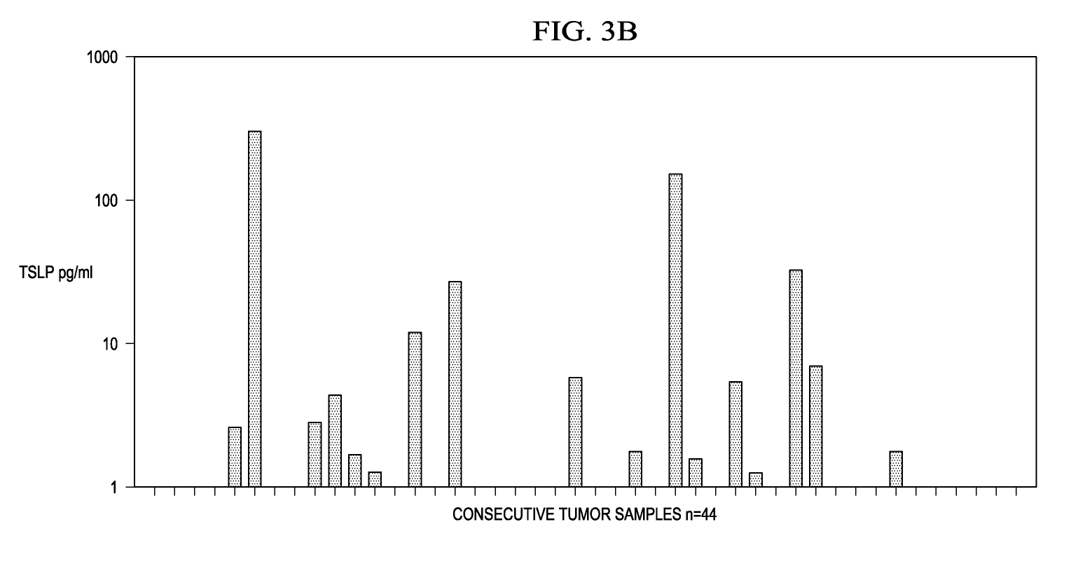

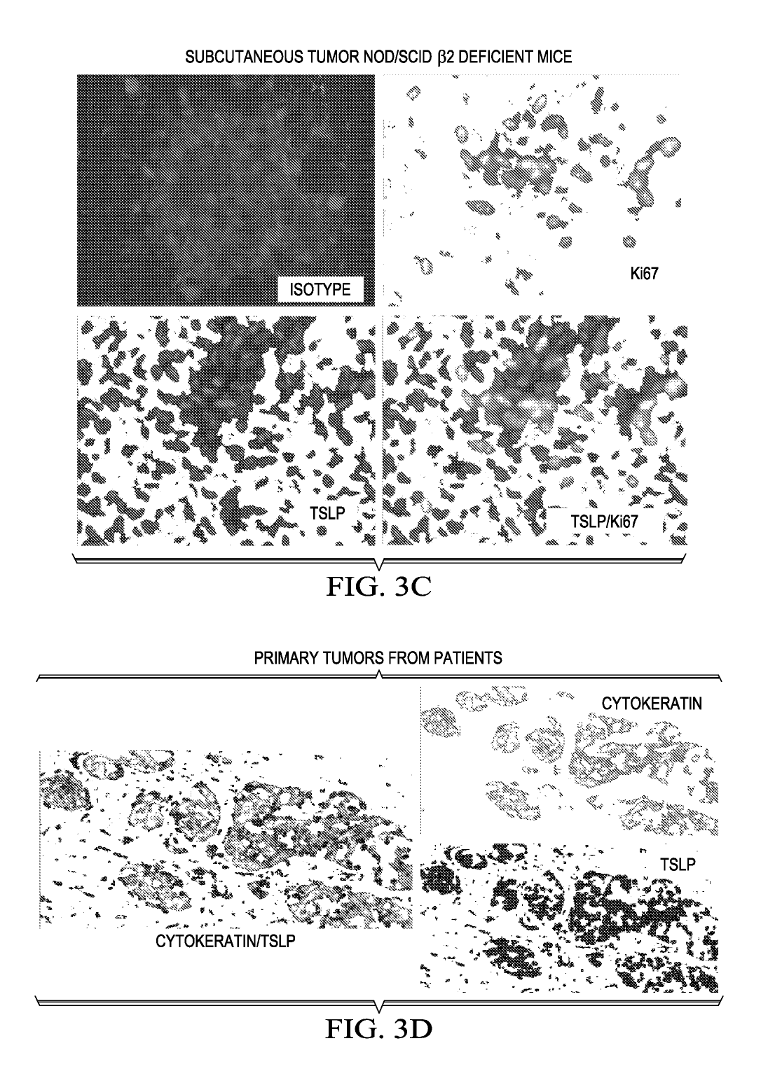

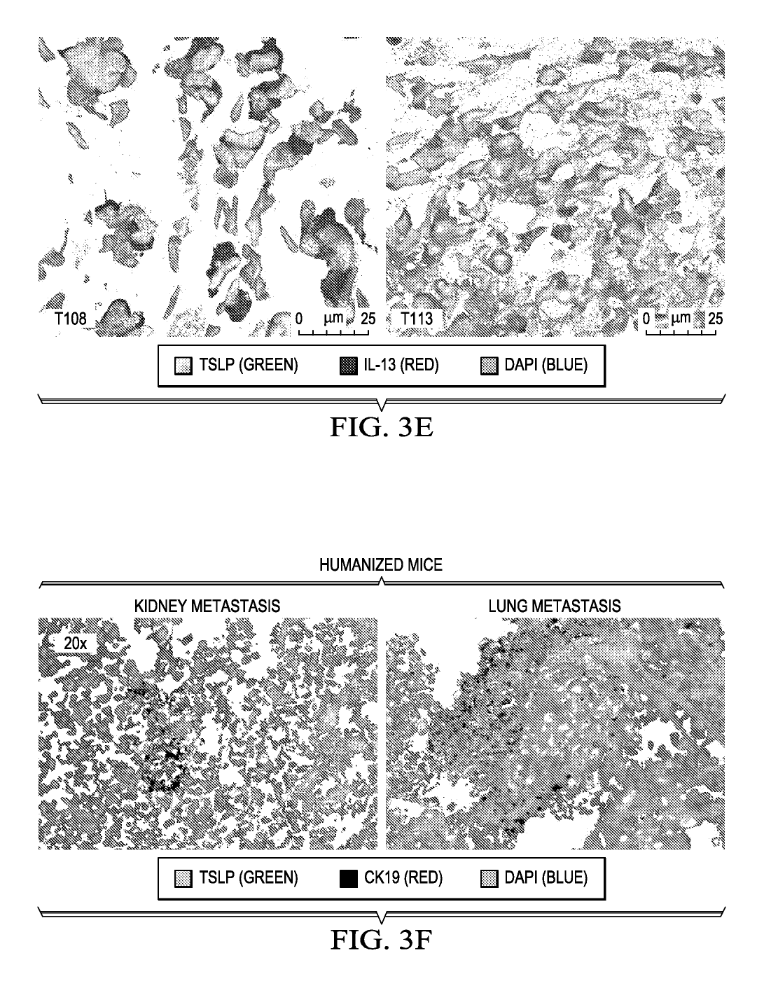

Breast cancer tumors express and secrete TSLP: OX40L can be induced on mDCs by TSLP, an IL-7 like cytokine produced by epithelial cells (Liu et al. 2007; Ziegler and Artis, 2010). The supernatants of the Hs578T breast cancer cell line contained low levels of TSLP, which could be substantially increased upon activation with PMA/Ionomycin (FIG. 3A). Supernatants of some primary breast cancer tumors activated with PMA/Ionomycin displayed up to 300 pg/ml TSLP (FIG. 3B). The expression of TSLP by cancer cells was further analyzed using an anti-TSLP antibody and immunofluorescence of frozen breast cancer tumors generated in the xenograft model (Aspord et al. 2007). There, subcutaneous MDA-MB-231 tumors transplanted in mice expressed TSLP (FIG. 3C). The specificity of the staining is demonstrated by pre-treatment of the antibody with recombinant TSLP.

Importantly, TSLP is expressed in 35 out of 38 analyzed primary breast cancer tumors obtained from patients regardless of grade, histology or stage of analyzed tumors. FIG. 3D illustrates the pattern of TSLP staining and co-expression with cytokeratin 19 positive cells. It demonstrates that TSLP is expressed in the cytoplasm and the nucleus of breast cancer cells that display IL-13 on their surface (FIG. 3E). Importantly, TSLP is also expressed in lung and kidney metastasis of MDA-MB-231 tumors in humanized mice (FIG. 3F) and in breast cancer tumor metastasis from patients.

Thus, similarly to normal skin or lung epithelium, breast cancer cells have the capacity to express, produce and secrete TSLP.

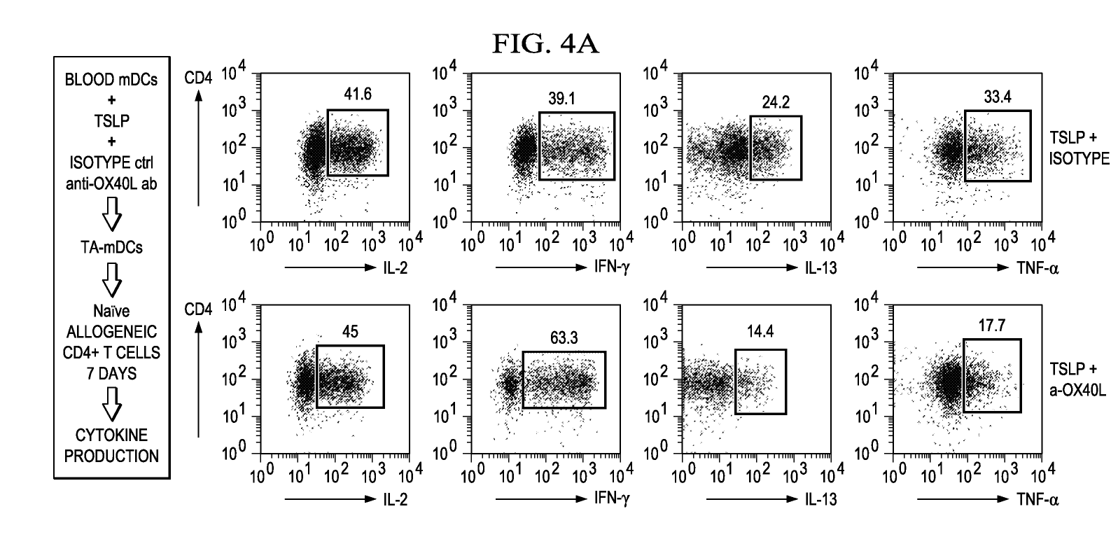

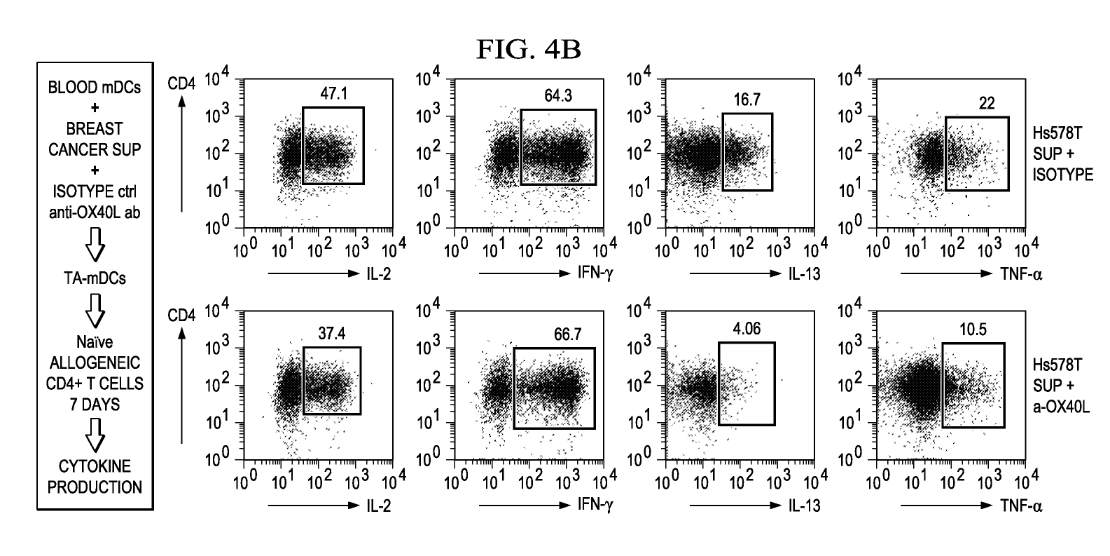

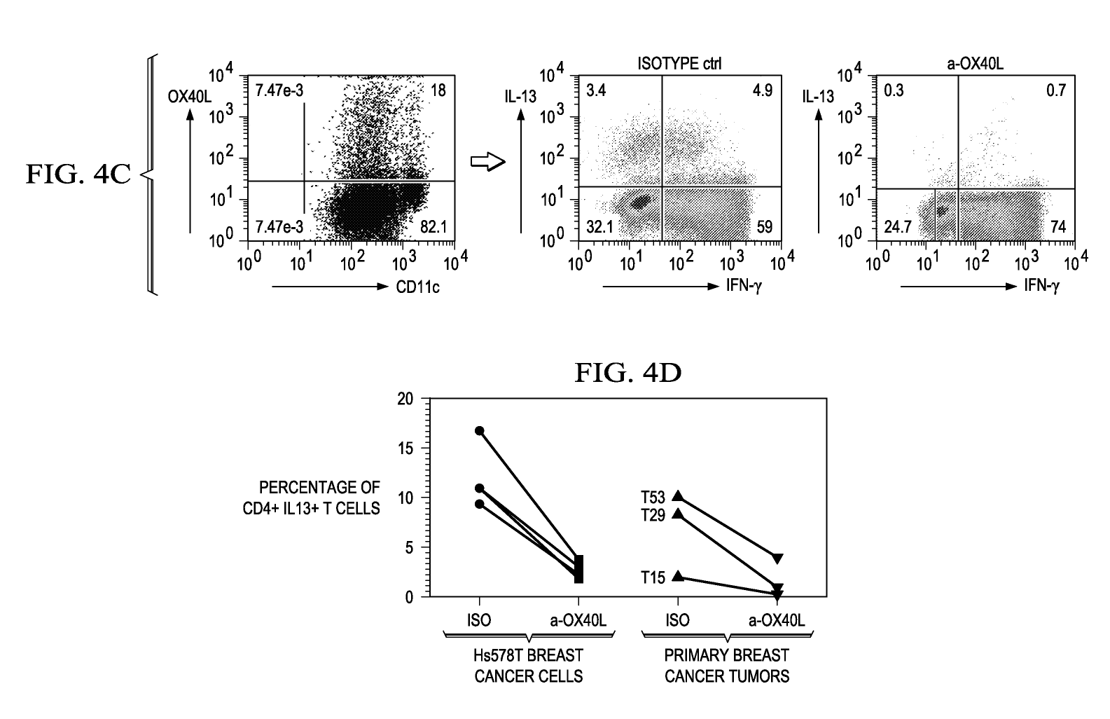

Anti-OX40L and anti-TSLP antibodies block the generation of inflammatory Th2 responses in vitro. To determine the impact of blocking TSLP and/or OX40L on the generation of inflammatory Th2 responses in breast cancer, blood mDCs were first exposed for 48 hours to either TSLP or breast tumor soluble fractions. Exposed mDCs were then used to stimulate naïve allogeneic CD4+ T cells with either the anti-OX40L antibody or a relevant isotype control. Blocking OX40L prevented the expansion of IL13+CD4+ or TNF+CD4+ T cells by 1) TSLP-primed mDCs (>50% inhibition, FIG. 4A), 2) mDCs exposed to Hs578T breast cancer cells (n=4, median inhibition of IL13+CD4+ cells=74%, range: 67-80%; FIGS. 4B and D), and 3) mDCs exposed to sonicates of randomly selected primary breast cancer tumors (FIGS. 4C and 4D).

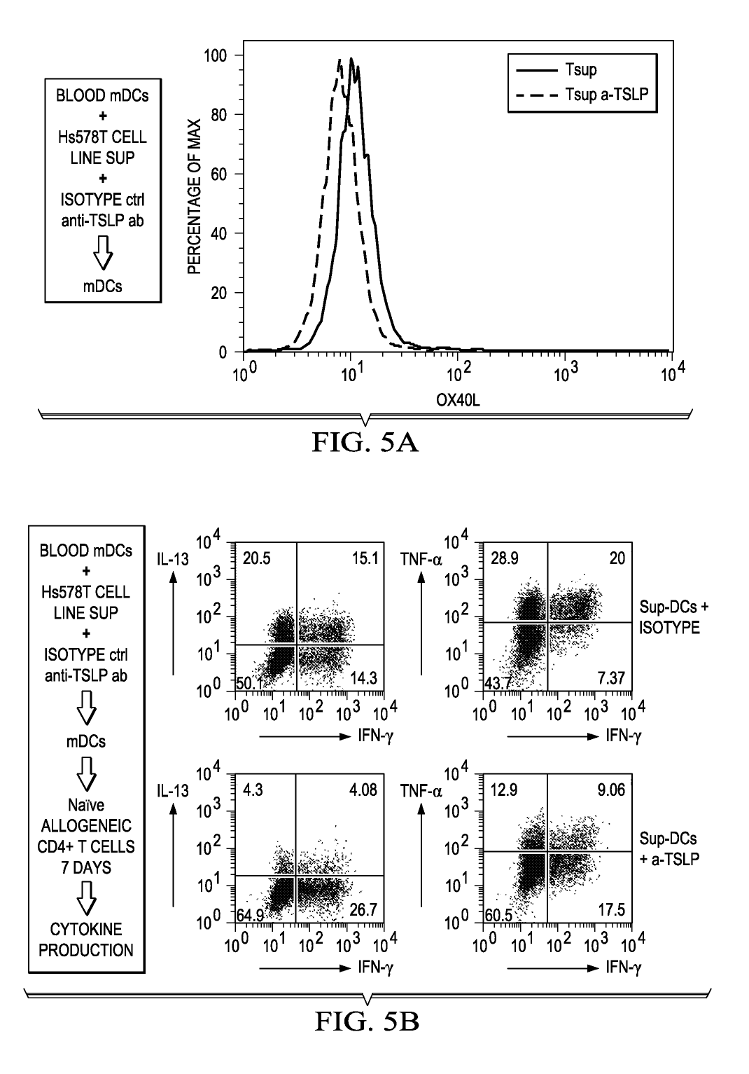

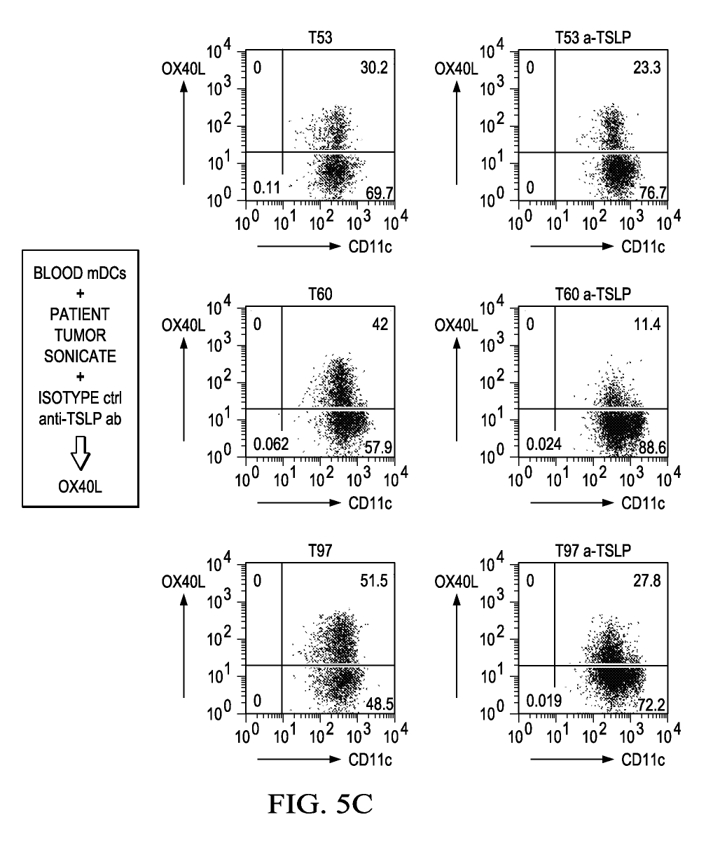

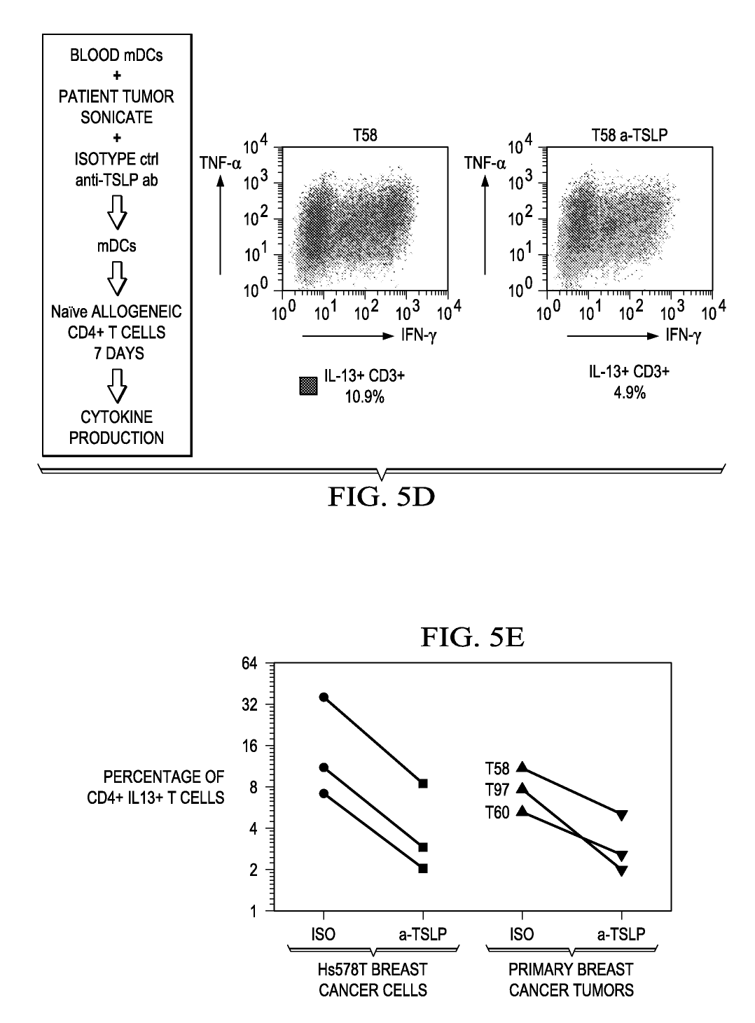

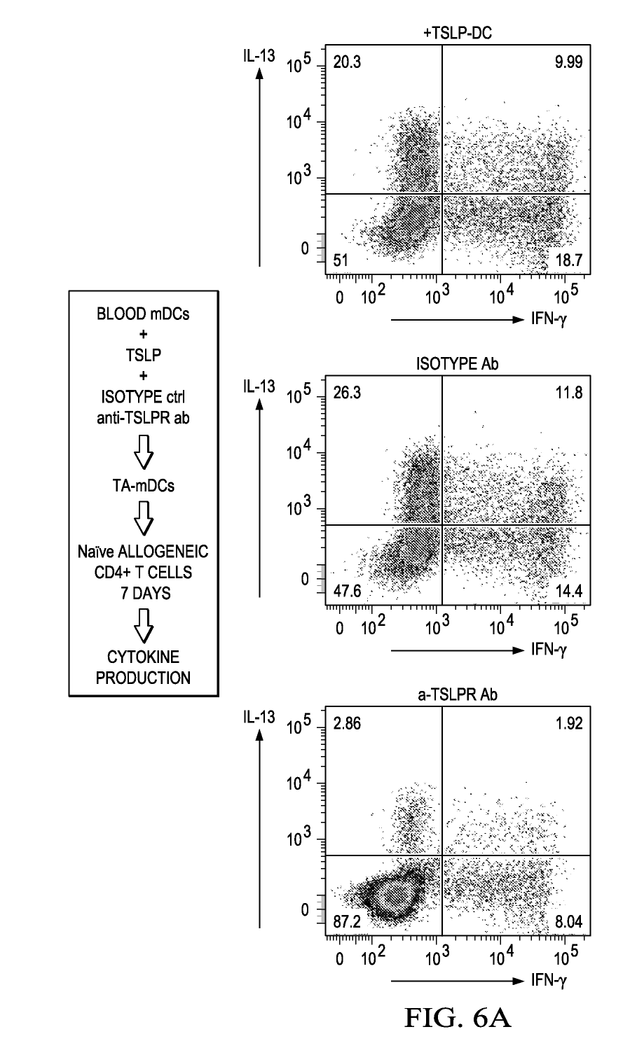

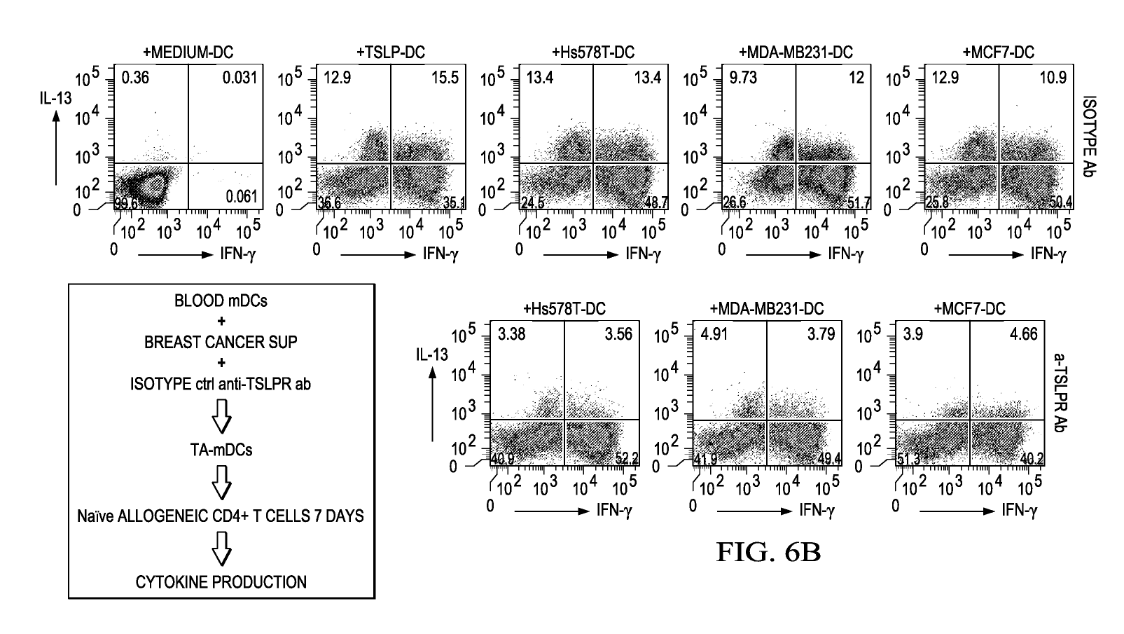

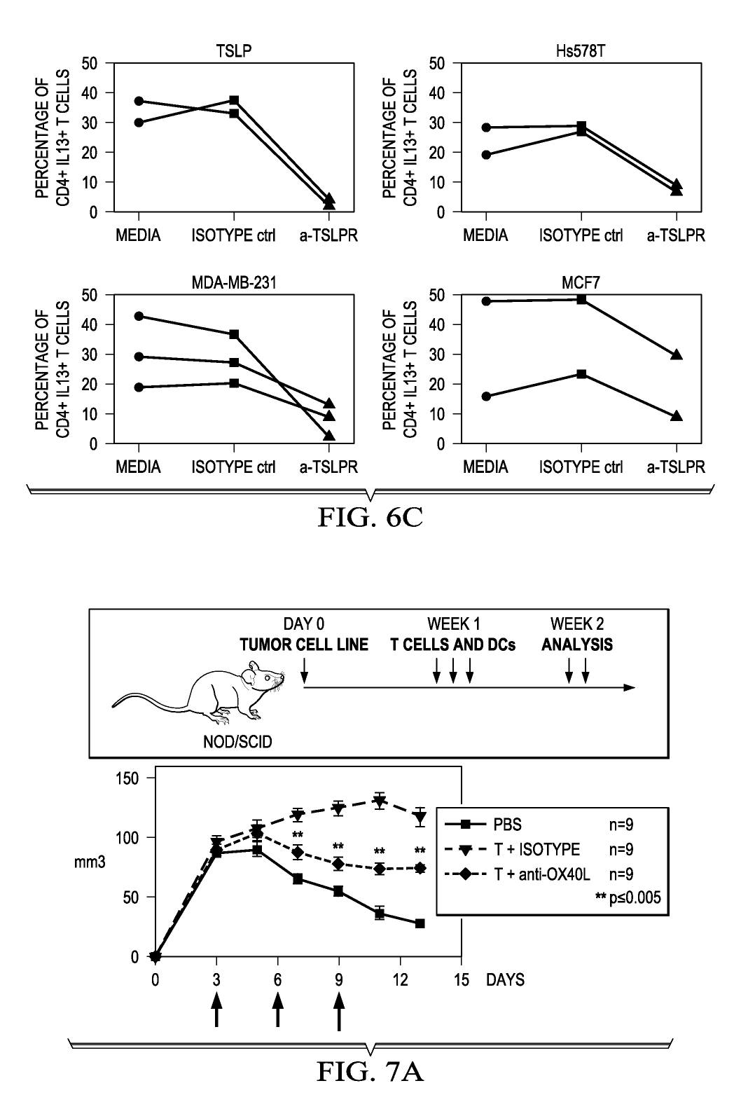

Addition of anti-TSLP neutralizing antibodies to breast cancer tumor supernatants inhibited their ability to induce OX40L on mDCs (FIG. 5A). Such mDCs displayed a diminished capacity to expand IL13+CD4+ or TNF+CD4+ T cells (n=3; median inhibition=73%, range: 72-77%; FIGS. 5B and 5E). Likewise, adding anti-TSLP neutralizing antibody sonicate of randomly selected primary breast cancer tumors led to down-regulation of OX40L expression by mDCs (FIG. 5C) and decreased expansion of IL13+TNF+CD4+ T cells (FIGS. 5D and 5E). Finally, when anti-TSLP receptor β chain (TSLPR) antibody was added to mDCs during their exposure to TSLP (FIG. 6A) and/or to the supernatant of three different breast cancer cell lines (Hs578T, MDA-MB231 and MCF7) (FIG. 6B), resulting mDCs showed a much-diminished expansion of IL13+CD4+ T cells (FIG. 6C). Thus, TSLP is the factor secreted by breast cancer cells, which contributes to generation of OX40L-dependent inflammatory Th2 responses.

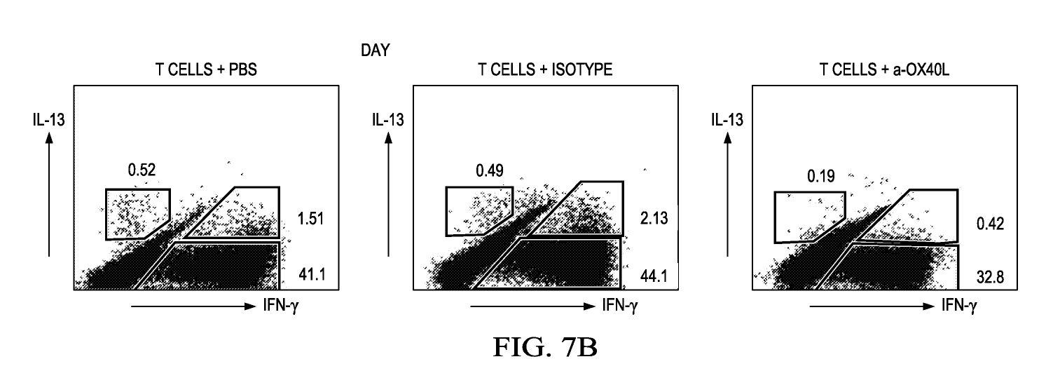

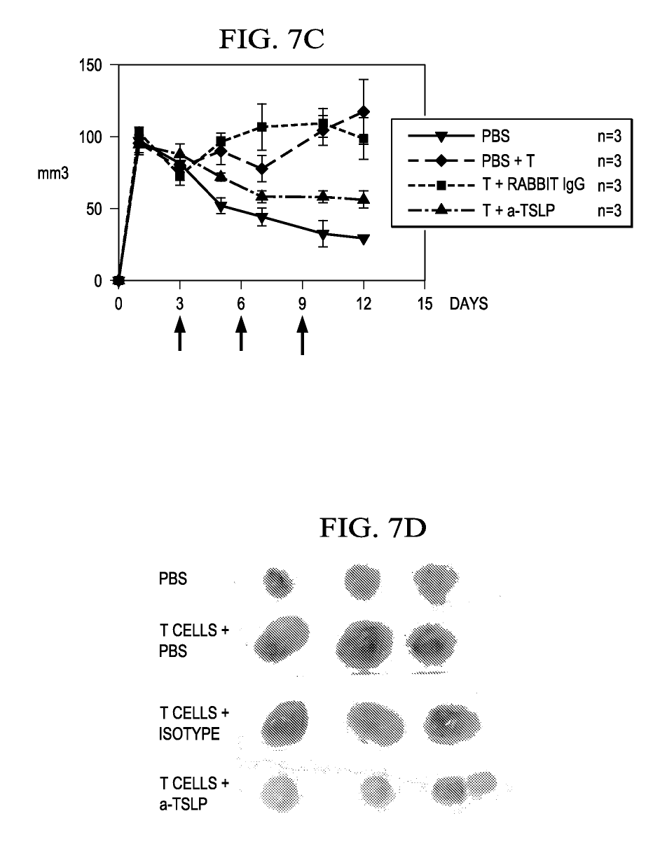

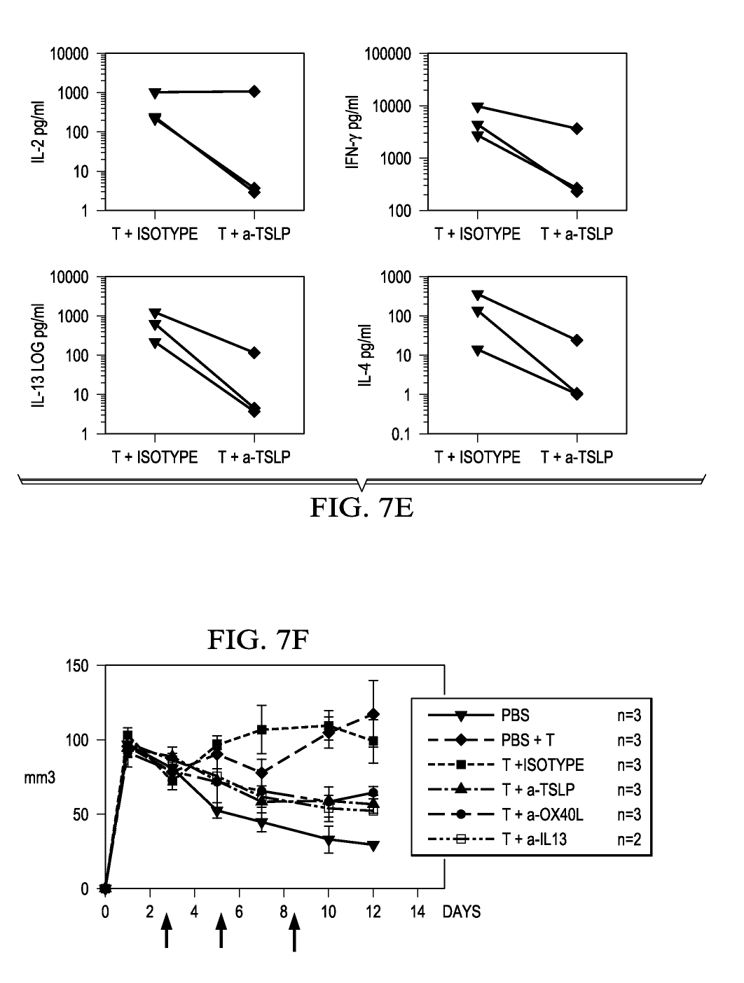

Antibodies neutralizing TSLP-OX40L axis block tumor development in vivo: Results of studies conducted in the present invention suggest a role for the TSLP-OX40L axis in generation of IL13+TNF+CD4+ T cells but do not establish whether this axis might actually contribute to breast cancer tumor development. To address this question, humanized mice were reconstituted with both Hs578T cells and T cells with or without anti-OX40L or anti-TSLP neutralizing antibodies (FIG. 7A shows the outline of experiments). As shown in FIG. 7A, the administration of neutralizing anti-OX40L antibodies leads to significant inhibition of tumor development that is associated with a decreased frequency of IL13+ CD4+ T cells at the tumor site (FIG. 7B).

The administration of a neutralizing anti-TSLP antibody also results in the inhibition of tumor development (FIG. 7C). This is illustrated by the tumors from three mice, which are smaller when measured both in the live animal (FIG. 7C) and after their surgical removal (FIG. 7D). TSLP blockade also leads to decreased secretion of IL-4 and IL-13 by tumor infiltrating T cells upon PMA/Ionomycin activation (FIG. 7E). Finally, when tested in a unique cohort of humanized mice the blockade of TSLP, OX40L and IL-13 resulted in a comparable inhibition of tumor growth (FIG. 7F). These results indicate that the TSLP-OX40L-IL-13 axis contributes to breast cancer pathogenesis.

There is accumulating evidence that inflammation plays a key role in the initiation and progression of cancer (reviewed in (Grivennikov et al. 2010)). There are two types of inflammation that have opposing effects on tumors: i) chronic inflammation that promotes cancer cell survival and metastasis (Condeelis and Pollard, 2006; Coussens and Werb, 2002; Mantovani et al. 2008); and ii) acute inflammation which can trigger cancer cell destruction as illustrated by regressions of bladder cancer following treatment with microbial preparations (Rakoff-Nahoum and Medzhitov, 2009). While chronic inflammation is often linked with the presence of type 2-polarized macrophages (M2), acute inflammation associated with cancer destruction is linked with type 1-polarized macrophages (M1). M1 macrophages are induced by the type 1 cytokine IFN-γ, whereas, M2 macrophages are induced by the type 2 cytokines, IL-4 and IL-13 (Alberto Mantovani and Sica, 2010)(50).

Like many other features of the immune response, Th1/Th2 polarization is regulated by DCs. The present inventors show herein that breast cancer is infiltrated with inflammatory Th2 cells and that such T cells are driven by OX40L on DCs. Blocking OX40L in vitro prevents generation of these CD4+ T cells without impact on IL-10 producing CD4+ T cells. Blocking OX40L in vivo partially prevents T cell-dependent acceleration of breast cancer tumor development. OX40L is not constitutively expressed but can be induced on DCs, macrophages and B cells for example upon CD40 engagement or cytokine signals such as TSLP or IL-18 as well as upon TLR stimulation (reviewed in (Croft et al. 2009)). Thus, the presence of OX40L+mDCs in breast tumors indicate sustained activation of DCs in tumor environment. Indeed, OX40L expression by DCs is driven by TSLP secreted from breast cancer cells. Accordingly, TSLP expression can be found in primary as well as metastatic tumors. Blocking TSLP reduces inflammation and partially inhibits tumor development.

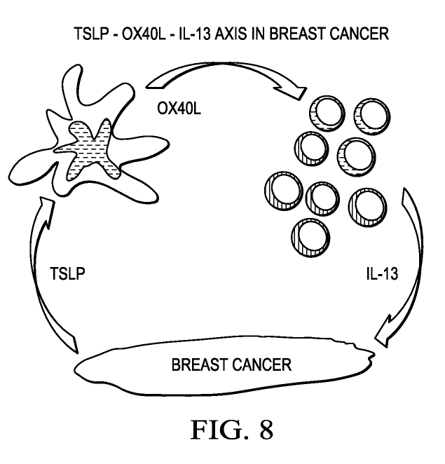

Based on our results presented herein and in our earlier studies the inventors propose a vicious circle of smoldering type 2 inflammation that perpetuates breast cancer and which is maintained by TSLP (FIG. 8). There, breast cancer attracts DCs possibly through macrophage inflammatory protein 3 alpha (MIP3-α) (Aspord et al. 2007; Bell et al. 1999). Tumor infiltrating DCs are then exposed to TSLP secreted by breast cancer cells, which triggers their maturation and OX40L expression. This might explain the aseptic mDC maturation as found in breast cancer (Aspord et al. 2007; Bell et al. 1999). OX40L+mDCs induce CD4+ T cells to secrete IL-13, as well as TNF-α. These inflammatory CD4⁺ T cells contribute to tumor development in an IL13-dependent pathway (FIG. 8). Thus far, TSLP represents the only factor that activates mDCs without inducing them to produce Th1-polarizing cytokines (Liu et al. 2007). Under normal physiological conditions, TSLP appears to play a critical role in CD4′ T cell homeostasis in the peripheral mucosa-associated lymphoid tissues and in the positive selection and/or expansion of Tregs in the thymus (Watanabe et al. 2005a; Watanabe et al. 2005b). In inflammatory conditions, such as atopic dermatitis and asthma, epithelial cells markedly increase TSLP expression (Liu et al. 2007). The TSLP-activated DCs migrate to the draining lymph nodes, prime CD4′ T cells via OX40L to differentiate into inflammatory Th2 effector and memory cells and therefore initiate the adaptive phase of allergic immune responses. Interestingly, in breast cancer OX40L⁺ mDCs are present in the tumor. It remains to be determined whether this reflects their inability to migrate from the tumor to draining lymph nodes. It also remains to be determined whether these DCs are able to prime Th2 immunity in situ in tertiary lymphoid structures or whether their main role is to maintain the activation and survival of Th2 cells at the tumor site. Their ability to maintain Th2 cell phenotype and effector function is supported by our earlier studies showing that T cells isolated from experimental breast tumors and transferred to naïve tumor bearing humanized mice can promote tumor development even at low numbers and upon single injection (Aspord et al. 2007).

The findings of the present invention add another feature to the role of OX40L in tumors. Indeed, a number of studies in mouse models of transplantable tumors suggested that engaging OX40 via an agonist antibody or OX40L.Fc or transfected tumor cells and DCs appears to promote anti-tumor effects (Ali et al. 2004; Morris et al. 2001; Piconese et al. 2008; Weinberg et al. 2000). However, Tnfsf4 is regulated by microRNA MIRN125B (Smirnov and Cheung, 2008) whose expression is downregulated in breast cancer (Iorio et al. 2005). Furthermore, Tnfsf4 is upregulated in ataxia-telangiectasia carriers and patients (Smirnov and Cheung, 2008) who have been shown having an increased risk of breast cancer (Swift et al. 1987). OX40L signaling has several important features that might help explain the results observed in our and other studies. Thus, OX40L triggers Th2 polarization independent of IL-4, promotes TNF production, and inhibits IL-10 production by the developing Th2 cells, but only in the absence of IL-12. In the presence of IL-12, OX40L signaling instead promotes the development of Th1 cells that, like inflammatory Th2 cells, produce TNF but not IL-10 (Liu et al. 2007).

Two key questions arise from the present invention: 1) what are the mechanisms allowing TSLP release from cancer cells; and 2) the impact of IL-13 (and IL-4) on cancer cells and on the immune infiltrate. For example, in the murine model of endogenous breast cancer CD4′ T cells contribute to the metastatic process via secretion of IL-4, which induces macrophages to secrete EGF (DeNardo et al. 2009). IL-13 can exert a pro-cancer activity in a number of ways including triggering of TGF-β secretion (Park et al. 2005; Shimamura et al. 2010; Terabe et al. 2000; Terabe et al. 2003). Furthermore, IL-4 exposure of cancer cells leads to the up-regulation of anti-apoptotic pathways via mobilization of STATE (Zhang et al. 2008). The inventors have previously shown earlier that STATE is phosphorylated in primary breast cancer tumors (Aspord et al. 2007). All these anti-apoptotic pathways are likely to synergize to promote the survival of cancer cell and facilitate metastasis. Importantly, such protective effect on cancer cells susceptibility to apoptosis might increase their resistance to chemotherapy (Todaro et al. 2008) as well as to immune-mediated cytotoxicity driven by Granzyme B (Heibein et al. 2000; Sarin et al. 1997). Thus, TSLP-OX40L-IL13 axis might offer a novel therapeutic target.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,709,217: Modified Human Thymic Stromal Lymphopoietin.
U.S. Patent Application Publication No. 20070237787: Methods for use of TSLP and Agonists and Antagonists Thereof
WIPO Patent Application No. WO/2005/007186: Treatment and Diagnosis of Neoplasms using Thymic Stromal Lymphopoietin.
U.S. Pat. No. 7,501,496: Anti-OX40L Antibodies.
U.S. Patent Application Publication No. 20100098712: Pharmaceutical Formulation of an Antibody against OX40L.
U.S. Patent Application Publication No. 20090053230: Anti-OX40L Antibodies and Methods using Same.
Albert, M. L., Darnell, J. C., Bender, A., Francisco, L. M., Bhardwaj, N., and Darnell, R. B. (1998). Tumor-specific killer cells in paraneoplastic cerebellar degeneration. Nat Med 4, 1321-1324.
Alberto Mantovani, and Sica, A. (2010). Macrophages, innate immunity and cancer: balance, tolerance, and diversity. Curr Opin Immunol.
Anderson, K. S. (2009). Tumor vaccines for breast cancer. Cancer Invest 27, 361-368.
Aspord, C., Pedroza-Gonzalez, A., Gallegos, M., Tindle, S., Burton, E. C., Su, D., Marches, F., Banchereau, J., and Palucka, A. K. (2007). Breast cancer instructs dendritic cells to prime interleukin 13-secreting CD4+ T cells that facilitate tumor development. J Exp Med 204, 1037-1047.
Banchereau, J., and Steinman, R. M. (1998). Dendritic cells and the control of immunity. Nature 392, 245-252.
Bell, D., Chomarat, P., Broyles, D., Netto, G., Harb, G. M., Lebecque, S., Valladeau, J., Davoust, J., Palucka, K. A., and Banchereau, J. (1999). In breast carcinoma tissue, immature dendritic cells reside within the tumor, whereas mature dendritic cells are located in peritumoral areas. J Exp Med 190, 1417-1426.
Berzofsky, J. A., and Terabe, M. (2008). A novel immunoregulatory axis of NKT cell subsets regulating tumor immunity. Cancer Immunol Immunother 57, 1679-1683.
Blanco, P., Palucka, A. K., Gill, M., Pascual, V., and Banchereau, J. (2001). Induction of dendritic cell differentiation by IFN-alpha in systemic lupus erythematosus. Science 294, 1540-1543.
Brimnes, M. K., Bonifaz, L., Steinman, R. M., and Moran, T. M. (2003). Influenza virus-induced dendritic cell maturation is associated with the induction of strong T cell immunity to a coadministered, normally nonimmunogenic protein. J Exp Med 198, 133-144.
Caux, C., Massacrier, C., Vanbervliet, B., Dubois, B., Durand, I., Cella, M., Lanzavecchia, A., and Banchereau, J. (1997). CD34+ hematopoietic progenitors from human cord blood differentiate along two independent dendritic cell pathways in response to granulocyte-macrophage colony-stimulating factor plus tumor necrosis factor alpha: II. Functional analysis. Blood 90, 1458-1470.
Condeelis, J., and Pollard, J. W. (2006). Macrophages: obligate partners for tumor cell migration, invasion, and metastasis. Cell 124, 263-266.
Coppola, D., and Mule, J. J. (2008). Ectopic lymph nodes within human solid tumors. J Clin Oncol 26, 4369-4370.
Coughlin, S. S., and Ekwueme, D. U. (2009). Breast cancer as a global health concern. Cancer Epidemiol 33, 315-318.
Coukos, G., Benencia, F., Buckanovich, R. J., and Conejo-Garcia, J. R. (2005). The role of dendritic cell precursors in tumour vasculogenesis. Br J Cancer 92, 1182-1187.
Coussens, L. M., and Werb, Z. (2002). Inflammation and cancer. Nature 420, 860-867.
Croft, M. (2003). Co-stimulatory members of the TNFR family: keys to effective T-cell immunity? Nat Rev Immunol 3, 609-620.
Croft, M., So, T., Duan, W., and Soroosh, P. (2009). The significance of OX40 and OX40L to T-cell biology and immune disease. Immunol Rev 229, 173-191.
Curiel, T. J., Cheng, P., Mottram, P., Alvarez, X., Moons, L., Evdemon-Hogan, M., Wei, S., Zou, L., Kryczek, I., Hoyle, G., et al. (2004). Dendritic cell subsets differentially regulate angiogenesis in human ovarian cancer. Cancer Res 64, 5535-5538.
Darnell, R. B. (1994). Paraneoplastic syndromes. in Current diagnosis in neurobiology E. Feldmann editor., 137-141.
Darnell, R. B. (1996). Onconeural antigens and the paraneoplastic neurologic disorders: at the intersection of cancer, immunity, and the brain. Proc Natl Acad Sci USA 93, 4529-4536.
DeNardo, D. G., Barreto, J. B., Andreu, P., Vasquez, L., Tawfik, D., Kolhatkar, N., and Coussens, L. M. (2009). CD4(+) T cells regulate pulmonary metastasis of mammary carcinomas by enhancing protumor properties of macrophages. Cancer Cell 16, 91-102.
Dieu-Nosjean, M. C., Antoine, M., Danel, C., Heudes, D., Wislez, M., Poulot, V., Rabbe, N., Laurans, L., Tartour, E., de Chaisemartin, L., et al. (2008). Long-term survival for patients with non-small-cell lung cancer with intratumoral lymphoid structures. J Clin Oncol 26, 4410-4417.

Disis, M. L., Bernhard, H., Gralow, J. R., Hand, S. L., Emery, S. R., Calenoff, E., and Cheever, M. A. (1994). Immunity to the HER-2/neu oncogenic protein. Ciba Found Symp 187, 198-207.
Disis, M. L., Bernhard, H., and Jaffee, E. M. (2009). Use of tumour-responsive T cells as cancer treatment. Lancet 373, 673-683.
Dudziak, D., Kamphorst, A. O., Heidkamp, G. F., Buchholz, V., Trumpfheller, C., Yamazaki, S., Cheong, C., Liu, K., Lee, H. W., Park, C. G., et al. (2007). Differential antigen processing by dendritic cell subsets in vivo. Science 315, 107-111.
Finkelman, F. D., Lees, A., Birnbaum, R., Gause, W. C., and Morris, S. C. (1996). Dendritic cells can present antigen in vivo in a tolerogenic or immunogenic fashion. J Immunol 157, 1406-1414.
Finn, O. J. (2008). Cancer immunology. N Engl J Med 358, 2704-2715.
Finn, O. J., Jerome, K. R., Henderson, R. A., Pecher, G., Domenech, N., Magarian-Blander, J., and Barratt-Boyes, S. M. (1995). MUC-1 epithelial tumor mucin-based immunity and cancer vaccines. Immunol Rev 145, 61-89.
Gilliet, M., Soumelis, V., Watanabe, N., Hanabuchi, S., Antonenko, S., de Waal-Malefyt, R., and Liu, Y. J. (2003). Human dendritic cells activated by TSLP and CD40L induce proallergic cytotoxic T cells. J Exp Med 197, 1059-1063.
Grivennikov, S. I., Greten, F. R., and Karin, M. (2010). Immunity, inflammation, and cancer. Cell 140, 883-899.
Hawiger, D., Inaba, K., Dorsett, Y., Guo, K., Mahnke, K., Rivera, M., Ravetch, J. V., Steinman, R. M., and Nussenzweig, M. C. (2001). Dendritic cells induce peripheral T cell unresponsiveness under steady state conditions in vivo. J Exp Med 194, 769-780.
Heibein, J. A., Goping, I. S., Barry, M., Pinkoski, M. J., Shore, G. C., Green, D. R., and Bleackley, R. C. (2000). Granzyme B-mediated cytochrome c release is regulated by the Bcl-2 family members bid and Bax. J Exp Med 192, 1391-1402.
Iorio, M. V., Ferracin, M., Liu, C. G., Veronese, A., Spizzo, R., Sabbioni, S., Magri, E., Pedriali, M., Fabbri, M., Campiglio, M., et al. (2005). MicroRNA gene expression deregulation in human breast cancer. Cancer Res 65, 7065-7070.
Ito, T., Wang, Y. H., Duramad, O., Hanabuchi, S., Perng, O. A., Gilliet, M., Qin, F. X., and Liu, Y. J. (2006). OX40 ligand shuts down IL-10-producing regulatory T cells. Proc Natl Acad Sci USA 103, 13138-13143.
Ito, T., Wang, Y. H., Duramad, O., Hori, T., Delespesse, G. J., Watanabe, N., Qin, F. X., Yao, Z., Cao, W., and Liu, Y. J. (2005). TSLP-activated dendritic cells induce an inflammatory T helper type 2 cell response through OX40 ligand. J Exp Med 202, 1213-1223.
Jaini, R., Kesaraju, P., Johnson, J. M., Altuntas, C. Z., Jane-Wit, D., and Tuohy, V. K. (2010). An autoimmune-mediated strategy for prophylactic breast cancer vaccination. Nat. Med.
Jones, K. L., and Buzdar, A. U. (2009). Evolving novel anti-HER2 strategies. Lancet Oncol 10, 1179-1187.
Klechevsky, E., Morita, R., Liu, M., Cao, Y., Coquery, S., Thompson-Snipes, L., Briere, F., Chaussabel, D., Zurawski, G., Palucka, A. K., et al. (2008). Functional specializations of human epidermal Langerhans cells and CD14+ dermal dendritic cells. Immunity 29, 497-510.
Levings, M. K., Gregori, S., Tresoldi, E., Cazzaniga, S., Bonini, C., and Roncarolo, M. G. (2005). Differentiation of Tr1 cells by immature dendritic cells requires IL-10 but not CD25+CD4+ Tr cells. Blood 105, 1162-1169.
Liu, Y. J. (2005). IPC: professional type 1 interferon-producing cells and plasmacytoid dendritic cell precursors. Annu Rev Immunol 23, 275-306.
Liu, Y. J. (2007). Thymic stromal lymphopoietin and OX40 ligand pathway in the initiation of dendritic cell-mediated allergic inflammation. J Allergy Clin Immunol 120, 238-244; quiz 245-236.
Liu, Y. J., Soumelis, V., Watanabe, N., Ito, T., Wang, Y. H., De Waal Malefyt, R., Omori, M., Zhou, B., and Ziegler, S. F. (2007). TSLP: An epithelial cell cytokine that regulates T cell differentiation by conditioning dendritic cell maturation. Annu Rev Immunol 25, 193-219.
Luft, T., Jefford, M., Luetjens, P., Toy, T., Hochrein, H., Masterman, K. A., Maliszewski, C., Shortman, K., Cebon, J., and Maraskovsky, E. (2002). Functionally distinct dendritic cell (DC) populations induced by physiologic stimuli: prostaglandin E(2) regulates the migratory capacity of specific DC subsets. Blood 100, 1362-1372.
Maldonado-Lopez, R., De Smedt, T., Michel, P., Godfroid, J., Pajak, B., Heirman, C., Thielemans, K., Leo, O., Urbain, J., and Moser, M. (1999). CD8alpha+ and CD8alpha− subclasses of dendritic cells direct the development of distinct T helper cells in vivo. J Exp Med 189, 587-592.
Mantovani, A., Romero, P., Palucka, A. K., and Marincola, F. M. (2008). Tumour immunity: effector response to tumour and role of the microenvironment. Lancet 371, 771-783.
Merad, M., Ginhoux, F., and Collin, M. (2008). Origin, homeostasis and function of Langerhans cells and other langerin-expressing dendritic cells. Nat Rev Immunol 8, 935-947.
Mohamadzadeh, M., Berard, F., Essert, G., Chalouni, C., Pulendran, B., Davoust, J., Bridges, G., Palucka, A. K., and Banchereau, J. (2001). Interleukin 15 skews monocyte differentiation into dendritic cells with features of Langerhans cells. J Exp Med 194, 1013-1020.
Nestle, F. O., Di Meglio, P., Qin, J. Z., and Nickoloff, B. J. (2009). Skin immune sentinels in health and disease. Nat Rev Immunol 9, 679-691.
Park, J. M., Terabe, M., van den Broeke, L. T., Donaldson, D. D., and Berzofsky, J. A. (2005). Unmasking immunosurveillance against a syngeneic colon cancer by elimination of CD4+NKT regulatory cells and IL-13. Int J Cancer 114, 80-87.
Pulendran, B., Smith, J. L., Caspary, G., Brasel, K., Pettit, D., Maraskovsky, E., and Maliszewski, C. R. (1999). Distinct dendritic cell subsets differentially regulate the class of immune response in vivo. Proc Natl Acad Sci USA 96, 1036-1041.
Rakoff-Nahoum, S., and Medzhitov, R. (2009). Toll-like receptors and cancer. Nat Rev Cancer 9, 57-63.
Reche, P. A., Soumelis, V., Gorman, D. M., Clifford, T., Liu, M., Travis, M., Zurawski, S. M., Johnston, J., Liu, Y. J., Spits, H., et al. (2001). Human thymic stromal lymphopoietin preferentially stimulates myeloid cells. J Immunol 167, 336-343.
Sallusto, F., and Lanzavecchia, A. (1994). Efficient presentation of soluble antigen by cultured human dendritic cells is maintained by granulocyte/macrophage colony-stimulating factor plus interleukin 4 and downregulated by tumor necrosis factor alpha. J Exp Med 179, 1109-1118.
Sarin, A., Williams, M. S., Alexander-Miller, M. A., Berzofsky, J. A., Zacharchuk, C. M., and Henkart, P. A. (1997). Target cell lysis by CTL granule exocytosis is independent of ICE/Ced-3 family proteases. Immunity 6, 209-215.
Shimamura, T., Fujisawa, T., Husain, S. R., Joshi, B., and Puri, R. K. (2010). Interleukin 13 mediates signal transduction through interleukin 13 receptor alpha2 in pancreatic ductal adenocarcinoma: role of IL-13 Pseudomonas exotoxin in pancreatic cancer therapy. Clin Cancer Res 16, 577-586.
Siegal, F. P., Kadowaki, N., Shodell, M., Fitzgerald-Bocarsly, P. A., Shah, K., Ho, S., Antonenko, S., and Liu, Y. J. (1999). The nature of the principal type 1 interferon-producing cells in human blood. Science 284, 1835-1837.
Smirnov, D. A., and Cheung, V. G. (2008). ATM gene mutations result in both recessive and dominant expression phenotypes of genes and microRNAs. Am J Hum Genet. 83, 243-253.
Soumelis, V., Reche, P. A., Kanzler, H., Yuan, W., Edward, G., Homey, B., Gilliet, M., Ho, S., Antonenko, S., Lauerma, A., et al. (2002). Human epithelial cells trigger dendritic cell mediated allergic inflammation by producing TSLP. Nat Immunol 3, 673-680.
Steinbrink, K., Jonuleit, H., G, M. l., Schuler, G., Knop, J., and Enk, A. H. (1999). Interleukin-10-treated human dendritic cells induce a melanoma-antigen-specific anergy in CD8(+) T cells resulting in a failure to lyse tumor cells. Blood 93, 1634-1642.
Steinman, R. M., and Banchereau, J. (2007). Taking dendritic cells into medicine. Nature 449, 419-426.
Steinman, R. M., Hawiger, D., and Nussenzweig, M. C. (2003). Tolerogenic dendritic cells. Annu Rev Immunol 21, 685-711.
Swift, M., Reitnauer, P. J., Morrell, D., and Chase, C. L. (1987). Breast and other cancers in families with ataxia-telangiectasia. N Engl J Med 316, 1289-1294.
Terabe, M., Matsui, S., Noben-Trauth, N., Chen, H., Watson, C., Donaldson, D. D., Carbone, D. P., Paul, W. E., and Berzofsky, J. A. (2000). NKT cell-mediated repression of tumor immunosurveillance by IL-13 and the IL-4R-STAT6 pathway. Nat Immunol 1, 515-520.
Terabe, M., Matsui, S., Park, J. M., Mamura, M., Noben-Trauth, N., Donaldson, D. D., Chen, W., Wahl, S. M., Ledbetter, S., Pratt, B., et al. (2003). Transforming growth factor-beta production and myeloid cells are an effector mechanism through which CD1d-restricted T cells block cytotoxic T lymphocyte-mediated tumor immunosurveillance: abrogation prevents tumor recurrence. J Exp Med 198, 1741-1752.
Todaro, M., Lombardo, Y., Francipane, M. G., Alea, M. P., Cammareri, P., Iovino, F., Di Stefano, A. B., Di Bernardo, C., Agrusa, A., Condorelli, G., et al. (2008). Apoptosis resistance in epithelial tumors is mediated by tumor-cell-derived interleukin-4 Cell Death Differ 15, 762-772.
Treilleux, I., Blay, J. Y., Bendriss-Vermare, N., Ray-Coquard, I., Bachelot, T., Guastalla, J. P., Bremond, A., Goddard, S., Pin, J. J., Barthelemy-Dubois, C., and Lebecque, S. (2004). Dendritic cell infiltration and prognosis of early stage breast cancer. Clin Cancer Res 10, 7466-7474.
Walzer, T., Dalod, M., Robbins, S. H., Zitvogel, L., and Vivier, E. (2005). Natural-killer cells and dendritic cells: “l′ union fait la force”. Blood 106, 2252-2258.
Watanabe, N., Hanabuchi, S., Marloie-Provost, M. A., Antonenko, S., Liu, Y. J., and Soumelis, V. (2005a). Human TSLP promotes CD40 ligand-induced IL-12 production by myeloid dendritic cells but maintains their Th2 priming potential. Blood 105, 4749-4751.
Watanabe, N., Wang, Y. H., Lee, H. K., Ito, T., Cao, W., and Liu, Y. J. (2005b). Hassall's corpuscles instruct dendritic cells to induce CD4+CD25+ regulatory T cells in human thymus. Nature 436, 1181-1185.
Wei, S., Kryczek, I., Zou, L., Daniel, B., Cheng, P., Mottram, P., Curiel, T., Lange, A., and Zou, W. (2005). Plasmacytoid dendritic cells induce CD8+ regulatory T cells in human ovarian carcinoma. Cancer Res 65, 5020-5026.
Zaba, L. C., Fuentes-Duculan, J., Steinman, R. M., Krueger, J. G., and Lowes, M. A. (2007). Normal human dermis contains distinct populations of CD11c+BDCA-1+ dendritic cells and CD163+FXIIIA+macrophages. J Clin Invest 117, 2517-2525.
Zhang, W. J., Li, B. H., Yang, X. Z., Li, P. D., Yuan, Q., Liu, X. H., Xu, S. B., Zhang, Y., Yuan, J., Gerhard, G. S., et al. (2008). IL-4-induced Stat6 activities affect apoptosis and gene expression in breast cancer cells. Cytokine 42, 39-47.
Ziegler, S. F., and Artis, D. (2010). Sensing the outside world: TSLP regulates barrier immunity. Nat Immunol 11, 289-293.

References Table 1.
Hackett A J, et al. Two syngeneic cell lines from human breast tissue: the aneuploid mammary epithelial(Hs578T) and the diploid myoepithelial (Hs578Bst) cell lines. J Natl Cancer Inst 58: 1795-1806, 1977;
Soule H D, et al. A human cell line from a pleural effusion derived from a breast carcinoma. J Natl Cancer Inst 51: 1409-1416, 1973;
Marc Lacroix and Guy Leclercq Relevance of breast cancer cell lines as models for breast tumours: an update. Breast Cancer Research and Treatment 83: 249-289, 2004.
Surmacz E. Function of the IGF-IR in breast cancer. J. Mammary Gland Biol. Neopl., 5: 95-105, 2000;
Monica Bartucci, et al. Differential Insulin-like Growth Factor I Receptor Signaling and Function in Estrogen Receptor (ER)-positive MCF-7 and ER-negative MDA-MB-231 Breast Cancer Cells. Cancer Research 61, 6747-6754, Sep. 15, 2001
www.ATCC.org
Valmori D, Fonteneau J F, Lizana C M, Gervois N, Lienard D, Rimoldi D, Jongeneel V, Jotereau F, Cerottini J C, Romero P (1998) Enhanced generation of specific tumor-reactive CTL in vitro by selected Melan-A/Mart-1 immunodominant peptide analogues. J Immunol 160:1750-1758
www.path.cam.ac.uk/˜pawefish/BreastCellLineDescriptions/HCC1806.html
Leah N. Klapper, et al (1999) The ErbB-2/HER2 oncoprotein of human carcinomas may function solely as a shared coreceptor for multiple stroma-derived growth factors. Cell Biology. Vol. 96, 4995-5000.
Neeraj K. Saxena, et al. Bidirectional Crosstalk between Leptin and Insulin-like Growth Factor-I Signaling Promotes Invasion and Migration of Breast Cancer Cells via Transactivation of Epidermal Growth Factor Receptor. Cancer Research 68, 9712, Dec. 1, 2008
Danica L. Rowe, et al. Modulation of the BRCA1 protein and induction of apoptosis in triple negative breast cancer cell lines by the polyphenolic compound curcumin.

What is claimed is:

1. A therapeutic composition for the treatment of a tumor of epithelial origin in a human subject comprising:
one or more active agents that bind and neutralize the activity of a thymic stromal lymphopoietin (TSLP), wherein the one or more active agents are selected from the group consisting of an anti-TSLP antibody; an anti-TSLP antibody fragment; an anti-TSLP antibody-carrier conjugate; a TSLP binding fusion protein; a TSLP antagonist; a TSLP inhibitor a TSLP receptor antagonist; or a TSLP blocking agent optionally solubilized, dispersed or suspended in a suitable medium in an amount sufficient to inhibit development of the tumor.

2. The composition of claim 1, wherein the composition is adapted to be administered intravenously; intramuscularly; subcutaneously; intraperitoneally; or parenterally.

3. The composition of claim 1, wherein the composition reduces a tumor-induced inflammation, inhibits tumor development, or both.

4. The composition of claim 1, wherein the composition decreases a level of IL-4, IL-13, or both.

5. The composition of claim 1, wherein the tumor is selected from a breast; prostate; kidney; lung; or pancreatic cancer.

6. The composition of claim 1, wherein the active agent decreases the infiltration of Th2 cells into the tumor.

7. The composition of claim 1, further comprising one or more pharmaceutically acceptable excipients.

8. The composition of claim 1, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

9. A method of treating or ameliorating symptoms of a cancer of epithelial origin in a human subject comprising the steps of:
identifying the subject in need of treatment against the cancer; and
administering a therapeutically effective amount of a pharmaceutical composition sufficient to treat or ameliorate the symptoms of the cancer in the subject comprising one or more monoclonal or polyclonal antibodies selected from the group consisting of an anti-OX40L antibody, an anti-thymic stromal lymphopoietin (TSLP) antibody, or both.

10. The method of claim 9, wherein the composition further comprises at least one of an anti-IL-13, anti-IL-2, anti-IL-4, anti-TNFα, and receptors thereof.

11. The method of claim 9, wherein the composition is administered intravenously; intramuscularly; subcutaneously; intraperitoneally; or parenterally.

12. The method of claim 9, wherein the composition reduces a tumor induced inflammation, inhibits tumor development, or both.

13. The method of claim 9, wherein the one or more antibodies are humanized.

14. The method of claim 9, wherein the composition further comprises one or more pharmaceutically acceptable excipients.

15. The method of claim 9, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

16. A composition for treating a cancer of epithelial origin in a patient comprising a therapeutically effective amount of one or more active agents that neutralize the activity of a thymic stromal lymphopoietin (TSLP) in the patient in an amount sufficient to reduce the cancer.

17. The composition of claim 16, wherein the one or more active agents are selected from the group consisting of an anti-TSLP antibody; an anti-TSLP antibody fragment; an anti-TSLP antibody-carrier conjugate; a TSLP binding fusion protein; a TSLP antagonist; a TSLP inhibitor; a TSLP receptor antagonist; or a TSLP blocking agent.

18. The composition of claim 17, wherein the anti-TSLP antibody is a humanized antibody.

19. The composition of claim 16, wherein the one or more active agents are dissolved, suspended or dispersed in suitable medium.

20. The composition of claim 16, wherein the one or more active agents that bind the TSLP decrease a level of an IL-4, IL-13 or both.

21. The composition of claim 16, wherein the one or more active agents reduce a tumor induced inflammation, inhibits tumor development or both.

22. The composition of claim 16, wherein the one or more active agents are adapted to be administered intravenously; intramuscularly; subcutaneously; intraperitoneally; or parenterally.

23. The composition of claim 16, wherein the cancer is selected from a breast; prostate; kidney; lung; or a pancreatic cancer.

24. The composition of claim 16, wherein the active agent decreases the infiltration of Th2 cells into a tumor of the cancer.

25. The composition of claim 16, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

26. A method of treating an individual who has a tumor, wherein the tumor secretes IL-13 comprising: administering to the individual a therapeutic composition comprising one or more agents that disrupt thymic stromal lymphopoietin (TSLP) activity selected from at least one of an anti-thymic stromal lymphopoietin (TSLP) antibody or antibody fragment; an anti TSLP protein; a TSLP antagonist; a TSLP inhibitor; a TSLP receptor antagonist; or TSLP blocking agent dispersed or solubilized in one or more optional pharmaceutically acceptable excipients.

27. The method of claim 26, wherein the anti-TSLP antibody is a humanized antibody.

28. The method of claim 26, wherein the composition is administered orally; intravenously; intramuscularly; subcutaneously; intraperitoneally; or parenterally.

29. The method of claim 26, wherein the composition reduces a tumor induced inflammation, inhibits tumor development, or both.

30. The method of claim 26, wherein the composition decreases a level of IL-4, IL-13, or both.

31. The method of claim 26, wherein the tumor comprises a breast; a prostate; a kidney; a lung; or a pancreatic cancer.

32. The method of claim 26, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

33. A therapeutic composition for the treatment of a tumor of epithelial origin in a human subject comprising one or more active agents that bind and neutralize the activity of a OX40 Ligand, wherein the one or more active agents are selected from the group consisting of an anti-OX40 Ligand antibody; an anti-OX40 Ligand antibody fragment; an anti-OX40 Ligand antibody-carrier conjugate; a OX40 Ligand binding fusion protein; a OX40 Ligand antagonist; a OX40 Ligand inhibitor; an OX40 receptor antagonist; or a OX40 Ligand blocking agent optionally solubilized, dispersed or suspended in a suitable medium in an amount sufficient to reduce development of the tumor.

34. The composition of claim 33, wherein the composition is adapted to be administered intravenously; intramuscularly; subcutaneously; intraperitoneally; or parenterally.

35. The composition of claim 33, wherein the composition reduces a tumor-induced inflammation, inhibits tumor development, or both.

36. The composition of claim 33, wherein the composition decreases a level of IL-4, IL-13, or both.

37. The composition of claim 33, wherein the tumor is selected from a breast; prostate; kidney; lung; or pancreatic cancer.

38. The composition of claim 33, wherein the active agent decreases the infiltration of Th2 cells into the tumor.

39. The composition of claim 33, further comprising one or more optional pharmaceutically acceptable excipients.

40. The composition of claim 33, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

41. A method of treating an individual who has a tumor, wherein the tumor secretes IL-13 comprising: administering to the individual, one or more active agents that disrupt OX40 Ligand selected from an anti-OX40 Ligand antibody; an anti-OX40 Ligand antibody fragment; an anti-OX40 Ligand antibody-carrier conjugate; a OX40 Ligand binding fusion protein; a OX40 Ligand antagonist; a OX40 Ligand inhibitor; or a OX40 Ligand blocking agent dispersed or solubilized in one or more optional pharmaceutically acceptable excipients.

42. The method of claim 41, wherein the antibody is a humanized antibody.

43. The method of claim 41, wherein the one or more active agents are administered intravenously; intramuscularly; subcutaneously; intraperitoneally; or by any other suitable parenteral route.

44. The method of claim 41, wherein the binding of the one or more active agents to the OX40 Ligand decrease a level of an IL-4, IL-13, or both.

45. The method of claim 41, wherein the tumor comprises a breast; a prostate; a kidney; a lung; or a pancreatic cancer.

46. The method of claim 41, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

47. A composition for treating a cancer of epithelial origin in a patient comprising a therapeutically effective amount of one or more active agents that neutralize the activity of an OX 40 Ligand in the patient in an amount sufficient to reduce the cancer.

48. The composition of claim 47, wherein the one or more active agents an anti-OX40 Ligand antibody; an anti-OX40 Ligand antibody fragment; an anti-OX40 Ligand antibody-carrier conjugate; a OX40 Ligand binding fusion protein; a OX40 Ligand antagonist; a OX40 Ligand inhibitor; or a OX40 Ligand blocking agent dispersed or solubilized in one or more optional pharmaceutically acceptable excipients.

49. The composition of claim 48, wherein the antibodies are humanized.

50. The composition of claim 47, wherein the composition treats the cancer by binding and neutralizing an OX40 Ligand.

51. The composition of claim 47, wherein the composition is administered orally; intravenously; intramuscularly; subcutaneously; intraperitoneally; or parenterally.

52. The composition of claim 47, wherein the composition reduces a tumor-induced inflammation, inhibits tumor development, or both.

53. The composition of claim 47, wherein the composition decreases a level of IL-4, IL-13, or both.

54. The composition of claim 47, wherein the tumor comprises a breast; a prostate; a kidney; a lung; or a pancreatic cancer.

55. The composition of claim 47, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.
